(19) United States
(12) Patent Application Publication
(10) Pub. No.: US 2024/0409605 A1
(43) Pub. Date: Dec. 12, 2024

(54) MULTI SUBUNIT PROTEIN MODULES, CELLS EXPRESSING SAME AND USES THEREOF

(71) Applicant: Ramot at Tel-Aviv University Ltd., Tel Aviv (IL)

(72) Inventors: Yaron CARMI, Tel Aviv (IL); Peleg RIDER, Tel Aviv (IL); Diana RASOULOUNIRIANA, Tel Aviv (IL); Lior TAL, Tel Aviv (IL)

(21) Appl. No.: 18/752,100

(22) Filed: Jun. 24, 2024

Related U.S. Application Data
(62) Division of application No. 18/085,632, filed on Dec. 21, 2022, now Pat. No. 12,077,569.
Application No. 18/085,632 is a continuation-in-part of application No. PCT/IL2021/050763, filed on Jun. 22, 2021.
(60) Provisional application No. 63/042,080, filed on Jun. 22, 2020.

(51) Int. Cl. C07K 14/735; A61K 39/00; A61P 37/04; C12N 5/0783

(52) U.S. Cl. CPC C07K 14/70535; A61K 39/4611; A61K 39/464404; A61K 39/464456; A61K 39/46449; A61K 2239/57; A61P 37/04; C12N 5/0636; C07K 2319/03; C07K 2319/30

(57) ABSTRACT

Multi subunit protein modules are provided. Accordingly, there is provided a multi subunit protein module comprising at least three cell membrane polypeptides each comprising an amino acid sequence of an Fc receptor common gamma chain (FcRgamma), said amino acid sequence is capable of transmitting an activating signal; wherein at least one but not all of said at least three polypeptides comprises an extracellular binding domain capable of binding a target that is presented on a cell surface of a target cell of an immune cell, such that upon binding of said extracellular binding domain to said target said activating signal is transmitted in an immune cell expressing said multi subunit protein module. Also provided are cells expressing the multi subunit protein modules and uses thereof.

### RELATED APPLICATIONS

This application is a divisional of U.S. patent application Ser. No. 18/085,632, filed December 21, 2022, which is a Continuation-in-Part (CIP) of International Application No. PCT/IL2021/050763 having an international filing date of Jun. 22, 2021, which claims the benefit of priority of U.S. Provisional Patent Application No. 63/042,080 filed on Jun. 22, 2020. The contents of the above applications are all incorporated by reference as if fully set forth herein in their entirety.

### SEQUENCE LISTING STATEMENT

The XML file, entitled 60957_702301_SequenceListing_v2.xml, created on Aug. 28, 2024, comprising 111,209 bytes, submitted concurrently with the filing of this application is incorporated herein by reference.

### FIELD AND BACKGROUND OF THE INVENTION

The present invention, in some embodiments thereof, relates to multi subunit protein modules, cells expressing same and uses thereof.

Cancer immunotherapy, including cell-based therapy, antibody therapy and cytokine therapy, has emerged in the last couple of years as a promising strategy for treating various types of cancer owing to its potential to evade genetic and cellular mechanisms of drug resistance and to target tumor cells while sparing healthy tissues.

Antibody-based cancer immunotherapies, such as monoclonal antibodies, antibody-fusion proteins, and antibody drug conjugates (ADCs) depend on recognition of cell surface molecules that are differentially expressed on cancer cells relative to non-cancerous cells and/or immune-checkpoint blockade. Binding of an antibody-based immunotherapy to a cancer cell can lead to cancer cell death via various mechanisms, e.g., antibody-dependent cell-mediated cytotoxicity (ADCC), complement-dependent cytotoxicity (CDC), direct cytotoxic activity of the payload from an antibody-drug conjugate (ADC) or suppressive checkpoint blockade. Many of these mechanisms initiate through the binding of the Fc domain of cell-bound antibodies to specialized cell surface receptors (Fc receptors) on hematopoietic cells.

Cell-based therapy using e.g. T cells having a T cell receptor (TCR) specific for an antigen differentially expressed in association with an MHC class I molecule on cancer cells relative to non-cancerous cells were shown to exert anti-tumor effects in several types of cancers, e.g. hematologic malignancies. However, antigen-specific effector lymphocytes, are very rare, individual-specific, limited in their recognition spectrum and difficult to obtain against most malignancies.

Strategies combining principles of antibody-based cancer immunotherapy and cell based therapy, such as CAR T cells and combined treatment with antibodies and T cells expressing Fc receptors have been disclosed (see e.g. EP Patent No: EP0340793; International Patent Application Publication No: WO2017205254 and WO2015179833; US Patent Application Publication Nos: US20150139943, US20180008638 and US20160355566; Clemenceau et al. Blood. 2006;107:4669-4677; and Urbanska et al. Molecular Therapy. 2014;22(Supplement 1): S297-S298). However, attempts made to date to harness these cells against solid tumors were disappointing. Thus, an urgent need to develop treatments capable of eradicating solid tumors, which feature a higher safety profile and do not depend exclusively on the host T-cell repertoire, still remains.

Additional background art includes Rasoulouniriana et al. J Clin Invest. (2019) 129(10): 4151-4164; U.S. Pat. Nos. 8,313,943 and 6,111,166; and International Patent Application Publication No: WO2015121454.

### SUMMARY OF THE INVENTION

According to an aspect of some embodiments of the present invention there is provided a multi subunit protein module comprising at least three cell membrane polypeptides each comprising an amino acid sequence of an Fc receptor common γ chain (FcRγ), the amino acid sequence is capable of transmitting an activating signal; wherein at least one but not all of the at least three polypeptides comprises an extracellular binding domain capable of binding a target that is presented on a cell surface of a target cell of an immune cell, such that upon binding of the extracellular binding domain to the target the activating signal is transmitted in an immune cell expressing the multi subunit protein module.

According to some embodiments of the invention, the binding domain is of a receptor and the target is a ligand of the receptor.

According to some embodiments of the invention, the binding domain is of an Fcγ receptor and the target is an Fc ligand.

According to some embodiments of the invention, the binding domain is of a ligand and the target is a receptor of the ligand.

According to some embodiments of the invention, the multi subunit protein module is devoid of an antibody binding domain.

According to some embodiments of the invention, the multi subunit protein module is devoid of an scFv.

According to some embodiments of the invention, the binding domain is of an antibody and the target is an antigen.

According to some embodiments of the invention, the binding domain comprises a scFv.

According to some embodiments of the invention, the at least one polypeptide comprising the binding domain comprises an amino acid sequence capable of recruiting polypeptides of the at least three polypeptides comprising the amino acid sequence of FcRγ comprised in the multi subunit protein module upon binding of the binding domain to the target.

According to some embodiments of the invention, the amino acid sequence capable of recruiting the other polypeptides comprises the transmembrane domain of an Fc receptor.

According to some embodiments of the invention, the Fc receptor is Fcγ receptor.

According to some embodiments of the invention, the Fcγ receptor is CD64.

According to some embodiments of the invention, at least two of the at least three cell membrane polypeptides comprise a dimerizing moiety.

According to some embodiments of the invention, the polypeptides comprising the amino acid sequence of FcRγ and not comprising the binding domain comprise a dimerizing moiety.

According to an aspect of some embodiments of the present invention there is provided a dimerizing moiety comprises an amino acid sequence of a transmembrane domain of the FcRγ.

According to some embodiments of the invention, the binding domain is of CD64 and the target is an Fc ligand; the at least one polypeptide comprising the binding domain comprises a transmembrane domain of CD64; and the polypeptides comprising the amino acid sequence of FcRγ and not comprising the binding domain comprise as a dimerizing moiety an amino acid sequence of a transmembrane domain of the FcRγ.

According to some embodiments of the invention, the target cell of the immune cell is a pathologic cell.

According to an aspect of some embodiments of the present invention there is provided at least one polynucleotide encoding the multi subunit protein module.

According to some embodiments of the invention, the at least one polynucleotide comprises a nucleic acid sequence encoding a first polypeptide comprising an extracellular and a transmembrane domain of CD64 and an intracellular domain of FcRγ; and a nucleic acid sequence encoding a second polypeptide comprising an extracellular, a transmembrane and an intracellular domain of FcRγ.

According to some embodiments of the invention, the multi subunit protein module is encoded by a single polynucleotide.

According to some embodiments of the invention, the polynucleotide comprises a nucleic acid sequence encoding a 2A skipping peptide between the nucleic acid sequence encoding the first polypeptide and the nucleic acid sequence encoding the second polypeptide.

According to an aspect of some embodiments of the present invention there is provided an immune cell genetically engineered to express the at least one polynucleotide.

According to an aspect of some embodiments of the present invention there is provided an immune cell expressing the at least one polynucleotide.

According to an aspect of some embodiments of the present invention there is provided an immune cell expressing the multi subunit protein module.

According to an aspect of some embodiments of the present invention there is provided a method of expressing a multi subunit protein module in an immune cell, the method comprising introducing into an immune cell the at least one polynucleotide, under conditions which allow expression of the multi subunit protein module.

According to some embodiments of the invention, the introducing is effected in-vitro or ex-vivo.

According to some embodiments of the invention, the immune cell is a T cell.

According to some embodiments of the invention, the immune cell is a NK cell.

According to an aspect of some embodiments of the present invention there is provided a T cell expressing a polypeptide complex, wherein the polypeptide complex comprises at least a first polypeptide and a second polypeptide, wherein the first and second polypeptides are not translationally fused, wherein the first polypeptide comprises an amino acid sequence of an Fc receptor common γ chain (FcRγ), the amino acid sequence is capable of transmitting an activating signal and forming a homodimer; and the second polypeptide comprising:
(i) an extracellular ligand-binding domain of an Fcγ receptor capable of binding an Fc ligand,
(ii) an amino acid FcRγ capable of transmitting an activating signal and
(iii) an amino acid sequence capable of recruiting the first polypeptide,
   such that upon binding of the Fc ligand to the extracellular ligand-binding domain of the Fcγ receptor the activating signal is transmitted.

According to an aspect of some embodiments of the present invention there is provided a method of treating a disease associated with a pathologic cell in a subject in need thereof, the method comprising administering to the subject a therapeutically effective amount of the immune cell, wherein the pathologic cell presents the target on its cell surface, thereby treating the disease in the subject.

According to an aspect of some embodiments of the present invention there is provided the immune cell, for use in treating a disease associated with a pathologic cell in a subject in need thereof, wherein the pathologic cell presents the target on its cell surface.

According to some embodiments of the invention, the subject is treated with a therapeutic composition comprising the target, the therapeutic composition being specific for the pathologic cell.

According to some embodiments of the invention, the method comprises administering to the subject a therapeutically effective amount of a therapeutic composition comprising the target, the therapeutic composition being specific for the pathologic cell.

According to an aspect of some embodiments of the present invention there is provided the immune cell and a therapeutic composition comprising the target, for use in treating a disease associated with a pathologic cell in a subject in need thereof, wherein the therapeutic composition being specific for the pathologic cell.

According to an aspect of some embodiments of the present invention there is provided an article of manufacture comprising a packaging material packaging the immune cells and a therapeutic composition comprising the target.

According to some embodiments of the invention, the therapeutic composition is specific for a pathologic cell.

According to some embodiments of the invention, wherein when the target is an Fc ligand, the therapeutic composition is an Fc-fusion protein.

According to some embodiments of the invention, wherein when the target is an Fc ligand, the therapeutic composition is an antibody.

According to some embodiments of the invention, the immune cell is a T cell; the binding domain is of CD64 and the target is an Fc ligand; and the therapeutic composition is an antibody specific for the pathologic cell.

According to an aspect of some embodiments of the present invention there is provided a method of increasing the killing capacity of an antibody against a pathologic cell in a subject in need thereof, the method comprising administering to the subject a therapeutically effective amount of:
(i) an antibody specific for the pathologic cell; and
(ii) the immune cell, wherein the immune cell is a T cell; wherein the binding domain is of CD64 and the target is an Fc ligand,
thereby increasing the killing capacity of the antibody against the pathologic cell.

According to some embodiments of the invention, the antibody is an IgG.

According to some embodiments of the invention, the pathologic cell is a cancerous cell and wherein the disease is cancer.

According to some embodiments of the invention, the cancer is selected from the group consisting of melanoma, lymphoma, colon cancer, lung cancer, breast cancer and pancreatic cancer.

According to some embodiments of the invention, the cancer is melanoma or lymphoma.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show schematic representations of the constructs used and illustrations of the receptors as expressed proteins. FIG. 1A demonstrates a construct encoding the extracellular, transmembrane and intracellular domains of FcγRI, T2A skipping peptide and FcRγ (SEQ ID NOs: 1-2), denoted herein as Alpha-2A-gamma. FIG. 1B demonstrates a construct encoding the extracellular and transmembrane domains of FcγRI fused to the intracellular domain of FcRγ, T2A skipping peptide and FcRγ (SEQ ID NOs: 3-4), denoted herein as Alpha-gamma 2A-gamma.

FIG. 2A-B show confocal microscopy of cells expressing the Alpha-2A-Gamma (FIG. 2A) or Alpha-gamma-2a-Gamma (FIG. 2B) construct and stained for TCRβ, FcγRI and GFP. ×200 magnitude.

FIG. 3 is a graph demonstrating the correlation between the number of cells counted by incuCyte imager in a field and the number of B16-H2B-tdTomato cells cultured in a well of 96 wells plate.

FIGS. 4A-B demonstrate killing of B16 target cells by Alpha-2A-Gamma infected cells in different ratios. FIG. 4A shows representative images taken by incuCyte imager following 2 days of co-culturing CD8+ T cells infected with Alpha-2A-Gamma and B16-H2B-tdTomato at the indicated effector:target ratios ranging from 0.5:1 to 16:1, in the presence of an anti-TRP-1 antibody. ×100 magnitude. FIG. 4B is a graph demonstrating the number of target cells counted by the incuCyte imager, following 2 days of co-culturing CD4+ or CD8+ T cells infected with Alpha-2A-Gamma and B16-H2B-tdTomato in different effector:target ratios with or without an anti-TRP-1 antibody.

FIGS. 5A, 5B and 5C demonstrate killing of B16 target cells by Alpha-gamma-2a-Gamma infected cells. FIG. 5A shows representative images taken by incuCyte imager following 2 days of co-culturing CD8+ T cells infected with Alpha-Gamma-2A-Gamma and B16-H2B-tdTomato at an effector:target ratio 2:1, in the presence or absence of an anti-TRP-1 antibody. ×100 magnitude. FIGS. 5B-C show graphs demonstrating the number of target cells counted by the incuCyte imager, following 2 days of co-culturing CD4+ (FIG. 5B) or CD8+ (FIG. 5C) T cells infected with Alpha-gamma-2A-Gamma at an effector:target ratio 2:1, in the presence or absence of an anti-TRP-1 antibody. Shown are also the following controls: B16 H2B-tdTomato target cells cultured alone, cultured with anti-TRP-1 antibody, co-cultured with non-infected CD4+ or CD8+ T cells (Sham), co-cultured with non-infected CD4+ or CD8+ T cells (Sham) and anti-TRP-1 antibody.

FIG. 6A-B show GFP and FcγRI (CD64) expression in infected effector T cells. FIG. 6A shows images taken following co-culturing B16-H2B-tdTomato with CD4+ or CD8+ T cells infected with Alpha-gamma-2A-Gamma and an anti-TRP-1 antibody, with bright light, red and green filters in incuCyte imager. ×200 magnitude. FIG. 6B shows flow cytometry analysis of non-infected (sham) or Alpha-gamma-2A-Gamma infected mouse CD4+ T cells, mouse CD8+ T cells or human Jurkat T cells. Cells were analyzed for FcγRI (CD64) staining and GFP expression.

FIG. 7 demonstrates killing of B16 target cells by Alpha-gamma-2a-Gamma infected cells. Shown are the number of B16-H2B-tdTomato target cells counted by the incuCyte imager, following 60 hours of co-culturing with CD4+ or CD8+ T cells infected with Alpha-gamma-2A-Gamma in the presence or absence of an anti-TRP-1 antibody, as compared to non-infected cells (sham). Also shown is a control of B16 H2B-tdTomato target cells cultured alone.

FIGS. 8A, 8B and 8C demonstrate the necessity of FcγRI receptor to signal through the dimer of the co-receptor FcRγ chain. FIG. 8A shows schematic representations of a construct composed of FcRI alpha chain extracellular D1-D3 domains fused to the hinge and transmembrane domains of CD8a together with signaling intracellular gamma (SEQ ID NOs: 5-6), denoted herein as Alpha-CD8-gamma; and a construct composed of the same sequence of Alpha-gamma-2A-Gamma construct, but with two cysteine residues in the transmembrane domain (Cysteine 25 and 44), mutated to glycine in order to prevent S—S bonds which facilitates dimerization (SEQ ID NOs: 7-8), denoted herein as Alpha-gamma-2A-Gamma (mut). FIG 8B shows representative images of B16-H2B-tdTomato target cells treated with an anti-TRP-1 antibody either alone or following 48 hours co-culturing with uninfected CD8+ T cells (Sham), CD8+ T cells infected with Alpha-2A-Gamma, Alpha-CD8-gamma, Alpha-gamma-2A-Gamma or Alpha-gamma-2A-Gamma (mut). Images were taken with bright light, red and green filters, ×200 magnitude. FIG. 8C is a graph showing target cell count following co-culturing with the CD8+ T cells described in FIG. 8B.

FIG. 9 demonstrates killing of YUMM1.7-H2B-tdTomato target cells by Alpha-gamma-2a-Gamma infected cells. Shown are representative images of YUMM1.7-H2B-tdTomato co-cultured for 2 days with Alpha-gamma-2A-Gamma infected CD8 T cells at an effector:target ratio 2:1, with or without an anti-TRP-1 antibody. Images were taken with bright light, red and green filters, showing target cells nuclear tdTomato and GFP in the infected T cells population.

FIG. 10A-B demonstrate killing of A20 B-cell lymphoma target cells by Alpha-gamma-2a-Gamma infected cells. FIG. 10A shows representative confocal images of A20 cells co-cultured with CD8+ T cells infected with Alpha-gamma-2A-Gamma cells at an effector:target ratio 2:1, with or without an anti-CD20 antibody. The cells were stained for T cell marker TCR and B cell marker B220. ×200 magnitude. FIG. 10B shows flow cytometry analysis of A20 B-lymphoma cells AnnexinV staining following 24 hours of co-culturing with Alpha-gamma-2A-Gamma CD8+ T cells, with or without an anti-CD20 antibody.

FIG. 11 demonstrates killing of B16 target cells by Alpha-gamma-2a-Gamma infected CD8+ T cells in combination with an anti-TRP-1 or with anti-CD44 antibody, following 24 hours of co-culturing.

FIG. 12 demonstrates the in-vivo anti-tumor effect of Alpha-gamma-2a-Gamma infected T cells. Shown is the tumor size in mice injected sub-cutaneously with B16 cells (2×10⁵) and treated with alpha-gamma-2A-Gamma infected CD3+ T cells with or without an anti-TRP-1 antibody (n=4 in each group), as compared to untreated (n=3) or treatment with non-infected (sham) CD3+ T cells and an anti-TRP-1 antibody (n=3) controls. Mice treatment started 7 days following tumor cell injection and comprised 3 injections on days 7, 11, 14. CD3+ T cells were injected i.v. while anti-TRP-1 was injected sub-cutaneously.

FIG. 13 shows a schematic representations of a construct encoding an anti-EGFR scFv fused to the extracellular D3 domain and transmembrane domain of FcγRI fused to the intracellular domain of FcRγ, T2A skipping peptide and FcRγ SEQ ID NOs: 37-38), denoted herein as scFv-alpha-gamma-2A-gamma.

FIGS. 14A, 14B, 14C, 14D, 14E, and 14F demonstrate killing of HER2 target cells by Alpha-gamma-2a-Gamma (AG2G) infected T cells as compared to Alpha-2A-Gamma (A2G) infected T cells. FIG. 14A shows representative flow cytometry plots of human T cells infected with AG2G-IRES-GFP retrovirus, 10 days following infection. FIG. 14B shows a representative confocal microscopy image of human T cells infected with AG2G-IRES-GFP and stained for FcγRI (CD64). FIG. 14C shows a representative confocal microscopy image of human HT29 colon cancer cell line infected with HER2 and H2b-tdTomato, and stained for HER2. FIG. 14D shows a representative confocal microscopy image of human HT29 colon cancer cell line infected with EGFR and H2b-tdTomato, and stained for EGFR. FIG. 14E is a graph demonstrating the number of target cells counted by the incuCyte imager of HT29-H2b-tdTomato HER2 cells co-cultured with uninfected (sham) or A2G-IRES-GFP or AG2G-IRES-GFP-infected human T cells and treated with the relevant anti-HER2 antibody Trastuzumab (n=4). FIG. 14F is a graph demonstrating the number of target cells counted by the incuCyte imager of HT29-H2b-tdTomato EGFR HT29 cells co-cultured with sham or A2G or AG2G-IRES-GFP-infected human T cells and treated with the irrelevant anti-HER2 antibody Trastuzumab (n=4).

FIGS. 15A, 15B, 15C, 15D, 15E and 15F demonstrate killing of HER2 target cells and EGFR target cells by T cells infected with Alpha-gamma-2a-Gamma (AG2G) pMSVG.1 vector in combination with the relevant antibody. FIG. 15A shows representative confocal microscopy images of T cells infected with the pMSVG.1-AG2G construct and stained for CD64. FIG. 15B is a graph demonstrating the number of target cells counted by the incuCyte imager of H2b-tdTomato HER2+ HT29 cells co-cultured with sham or AG2G-infected human T cells with or without treatment with the relevant anti-HER2 antibody Trastuzumab (n=4). FIG. 15C is a graph demonstrating the number of target cells counted by the incuCyte imager of H2b-tdTomato HER2+ HT29 cells co-cultured with sham or AG2G-infected human T cells with or without treatment with the irrelevant anti-EGFR antibody Cetuximab (n=4). FIGS. 15D-E show graphs demonstrating TNFα (FIG. 15D) and IFNγ (FIG. 15E) concentration in supernatants of H2b-tdTomato HER2+ HT29 human colon carcinoma co-cultured with sham or AG2G-infected human T cells at effector:target ratio of 4:1, 2:1 or 1:1 with or without treatment with the relevant anti-HER2 antibody Trastuzumab, as determined by ELISA. FIG. 15F is a graph demonstrating the number of target cells counted by the incuCyte imager of H2b-tdTomato HER2+ HT29 cells co-cultured with sham or AG2G-infected human T cells with or without treatment with the relevant anti-HER2 antibody Trastuzuman or the irrelevant antibodies, Cetuximab or Retuximab.

FIG. 16 shows schematic representations of costimulatory and cytokine receptor signaling domains that can be added to the polypeptide of some embodiments of the invention.

FIG. 17 shows GFP and FcγRI (CD64) expression in human γδT cells infected with the Alpha-gamma-2a-Gamma (AG2G)-IRES-GFP vector, 9 days following infection. Human PBMC from healthy donor were collected and γδ T cells isolated following expansion using Zoledronate and IL-2 were transduced with retrovirus for AG2G cloned in pMIGII vector, and were imaged for GFP expression and for γδ-TCR-APC or CD64-APC staining.

FIGS. 18A-B show CD56 and FcγRI (CD64) expression in NK-92 cell line infected with Alpha-gamma-2a-Gamma (AG2G) pMSVG.1 vector. FIG. 18A shows representative flow cytometry plot demonstrating expression of AG2G using an anti-CD64-APC antibody. FIG. 18B shows representative immunostaining images demonstrating expression of the NK marker CD56 using an anti-CD56-APC antibody and expression of AG2G using an anti-CD64-APC antibody.

### DESCRIPTION OF SPECIFIC EMBODIMENTS OF THE INVENTION

The present invention, in some embodiments thereof, relates to multi subunit protein modules, cells expressing same and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cancer immunotherapy, including cell-based therapy, antibody therapy and cytokine therapy, has emerged in the last couple of years as a promising strategy for treating various types of cancer. Antibody-based cancer immunotherapies depend on recognition of cell surface molecules that are differentially expressed on cancer cells relative to non-cancerous cells and/or immune-checkpoint blockade. On the other hand, cell-based therapy using e.g. T cells having a T cell receptor (TCR) specific for an antigen differentially expressed in association with an MHC class I molecule on cancer cells relative to non-cancerous cells were shown to exert anti-tumor effects in several types of cancers, e.g. hematologic malignancies. Strategies combining principles of antibody-based cancer immunotherapy and cell based therapy have been suggested.

Specific embodiments of the present teachings suggest that the formation of a three polypeptide structure each comprising an Fcγ chain activating domain, is surprisingly advantageous in comparison to a single polypeptide comprising an Fcγ chain activating domain or two polypeptides comprising an Fcγ chain (e.g. created by the dimerizing properties of the transmembrane domain of Fcγ chain).

As shown in the Examples section which follows, the present inventors have exogenously expressed in T cells a first polypeptide comprising the binding domain of the high affinity Fcγ receptor FcγRI (CD64) and a second distinct polypeptide comprising the Fcγ chain which is capable of forming a homodimer. These engineered T cells exerted in-vitro killing capabilities of tumor cells in combination with anti-tumor antibodies, which were advantageous to T cells exogenously expressing a single polypeptide comprising both the binding domain of FcγRI and the activating domain of Fcγ chain (Example 1 of the Examples section which follows). However, the present inventors discovered that by combining the two approaches and exogenously expressing a first polypeptide comprising both the binding domain of FcγRI and the activating domain of Fcγ chain and a second distinct polypeptide comprising Fcγ chain which is capable of forming a homodimer, the engineered T cells exerted more robust and in lower concentrations in-vitro killing capabilities (Example 1 of the Examples section which follows). Importantly, these cells also had a remarkable anti-tumor effect in an in-vivo mouse tumor model (Example 2 of the Examples section which follows).

Thus, according to an aspect of the present invention, there is provided a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface of a target cell of an immune cell, such that upon binding of said extracellular binding domain to said target said activating signal is transmitted in an immune cell expressing said multi subunit protein module.

As used herein, the phrase “multi subunit protein module” refers to a plurality of at least three polypeptides which together have the activity of transmitting an activating signal in an immune cell expressing the multi subunit protein module upon binding of the extracellular domain to its target presented on a cell surface of a target cell.

According to specific embodiments, the multi subunit protein module comprises 3, 4, 5, 6 or more polypeptides.

According to a specific embodiment, the multi subunit protein module comprises 3 polypeptides.

According to specific embodiments, the plurality of polypeptides are complexed (or assembled) together (e.g. following binding of the extracellular binding domain to its target).

According to specific embodiments, wherein said at least one polypeptide is one polypeptide.

According to specific embodiments, wherein said at least two polypeptides is two polypeptides.

According to specific embodiments, wherein said at least three polypeptides is three polypeptides.

As used herein the phrase “Fc receptor common γ chain” abbreviated as “FcRγ” refers to the polypeptide expression product of the FCER1G gene (Gene ID 2207). According to specific embodiments, FcRγ is human FcRγ. According to a specific embodiment, the FcRγ protein refers to the human protein, such as provided in the following GenBank Number NP_004097 (SEQ ID NO: 11).

As used herein, the phrase “amino acid sequence of an Fc receptor common γ chain (FcRγ) capable of transmitting an activating signal” refers to full length FcRγ or a fragment thereof or a to homolog thereof which comprises an intracellular domain and maintains at least the capability of transmitting an activating signal in a cell expressing an Fcγ receptor upon binding of the Fcγ receptor to a Fc ligand.

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

According to specific embodiments, the amino acid sequence of an Fc receptor common γ chain (FcRγ) capable of transmitting an activating signal comprises an ITAM motif.

As used herein the terms “activating” or “activation” refer to the process of stimulating a T cell that results in cellular proliferation, maturation, cytokine production, chemotaxis and/or induction of effector functions.

Methods of determining signaling of an activating signal are well known in the art, and include, but are not limited to, enzymatic activity assays such as kinase activity assays, and expression of molecules involved in the signaling cascade using e.g. PCR, Western blot, immunoprecipitation and immunohistochemistry. Additionally or alternatively, determining transmission of an activating signal can be effected by evaluating T cell activation or function. Methods of evaluating T cell activation or function are well known in the art and include, but are not limited to, proliferation assays such as CFSE staining, MTT, Alamar blue, BRDU and thymidine incorporation, cytotoxicity assays such as CFSE staining, chromium release, Calcin AM, cytokine secretion assays such as intracellular cytokine staining, ELISPOT and ELISA, expression of activation markers such as CD25, CD69, CD137, CD107a, PD1, and CD62L using flow cytometry.

According to specific embodiments, the amino acid sequence of FcRγ comprises full length FcRγ.

Thus, the polypeptide of some embodiments of the invention comprises full length FcRγ.

According to specific embodiments, the amino acid sequence of FcRγ comprises a fragment thereof or a homolog thereof which comprises an intracellular domain and maintains at least the capability of transmitting an activating signal in a cell expressing an Fcγ receptor upon binding of the Fcγ receptor to a Fc ligand.

The polypeptide of some embodiments of the invention comprises the intracellular domain of FcRγ capable of transmitting an activating signal and are devoid of the membranal and extracellular domains of FcRγ.

According to specific embodiments, the multi subunit protein module comprises at least one polypeptide comprising the intracellular domain of FcRγ capable of transmitting an activating signal and is devoid of the membranal and extracellular domains of FcRγ; and at least two polypeptides comprising full length FcRγ.

According to specific embodiments, the multi subunit protein module comprises a single polypeptide comprising the intracellular domain of FcRγ capable of transmitting an activating signal and is devoid of the membranal and extracellular domains of FcRγ; and two polypeptides comprising full length FcRγ.

According to specific embodiments, the amino acid sequence of FcRγ capable of transmitting an activating signal comprises SEQ ID NO: 12 or 39.

According to specific embodiments, the amino acid sequence of FcRγ capable of transmitting an activating signal consists of SEQ ID NO: 12 or 39.

The homolog (naturally occurring or synthetically/recombinantly produced) can be, for example, at least 50%, at least 55%, at least 60%, at least 65%, at least 70%, at least 75%, at least 80%, at least 85%, at least 90%, at least 95%, at least 96%, at least 97%, at least 98%, at least 99% or 100% identical or homologous to the polypeptide provided in SEQ ID NO: 11, 12 or 39 or a functional fragment thereof which exhibit the desired activity (i.e., comprises an intracellular domain and maintains at least the capability of transmitting an activating signal in a cell expressing an Fcγ receptor upon binding of the Fcγ receptor to a Fc ligand); or at least 50%, at least 55%, at least 60%, at least 65%, at least 70%, at least 75%, at least 80%, at least 85%, at least 90%, at least 95%, at least 96%, at least 97%, at least 98%, at least 99% or 100% identical to the polynucleotide sequence encoding same.

The homolog (naturally occurring or synthetically/recombinantly produced) can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide provided in SEQ ID NO: 11, 12 or 39 or a functional fragment thereof which exhibit the desired activity (i.e., comprises an intracellular domain and maintains at least the capability of transmitting an activating signal in a cell expressing an Fcγ receptor upon binding of the Fcγ receptor to a Fc ligand); or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including a conservative and non-conservative amino acid substitution, as further described hereinbelow.

According to specific embodiments, the amino acid sequence of FcRγ may comprise conservative and/or non-conservative amino acid substitutions.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)₅—COOH]—CO— for aspartic acid. Those non-conservative substitutions which fall under the scope of the present invention are those which still constitute an amino acid sequence capable of binding α4β7 integrin.

At least one but not all of the at least three cell membrane polypeptides comprised in the multi subunit protein module disclosed herein comprises an extracellular binding domain capable of binding a target that is presented on a cell surface of a target cell of an immune cell.

According to specific embodiments, one of the at least three cell membrane polypeptides comprised in the multi subunit protein module disclosed herein comprises an extracellular binding domain capable of binding a target that is presented on a cell surface of a target cell of an immune cell.

Thus, according to specific embodiments, the cell membrane polypeptide comprises an extracellular binding domain capable of binding a target that is presented on a cell surface of a target cell of an immune cell.

According to other specific embodiments, the cell membrane polypeptide is devoid of an extracellular binding domain capable of binding a target that is presented on a cell surface of a target cell of an immune cell.

According to a specific embodiment, the multi subunit protein module comprises three cell membrane polypeptides each comprising an amino acid sequence of an FcRγ, wherein one of the cell membrane polypeptides further comprises an extracellular binding domain capable of binding a target that is presented on a cell surface of a target cell of an immune cell and the other two cell membrane polypeptides are devoid of such an extracellular binding domain.

As used herein, the phrase “extracellular binding domain capable of binding a target” refers to a proteinaceous moiety having a binding affinity (e.g., below 10⁻⁴ nM) to a target of interest being presented on a target cell. Non-limiting examples of binding domains include the binding domain of a receptor, the binding domain of a ligand, the binding domain of a hormone (e.g. leptin) and an antigen binding moiety such an antibody, as further described hereinbelow.

Assays for testing binding are well known in the art and include, but not limited to flow cytometry, bio-layer interferometry Blitz® assay, HPLC, surface plasmon resonance (e.g. Biacore).

According to specific embodiments, the extracellular binding domain of binds the target presented on the cell with a Kd>10⁻⁶ M, >10⁻⁷ M, >10⁻⁸ M or >10⁻⁹ M, each possibility represents a separate embodiment of the present invention.

According to specific embodiments, the extracellular binding domain binds the target presented on the cell with a Kd>10⁻⁹ M.

As used herein, the phrase “target cell of an immune cell” refers to a cell which upon recognition by an immune cell causes activation of the immune cell via the multi subunit protein module described herein.

According to specific embodiments, the target cell is a pathologic (diseased) cell.

According to specific embodiments, the target cell is a cancerous cell.

As used herein, the term “presented” refers to a target expressed by the target cell (e.g. an antigen) or bound to (yet not expressed by it) the target cell (e.g. an antibody which binds an antigen expressed by the target cell).

According to specific embodiments, the target is over-presented or only presented on the cell surface of the target cells as compared to other cells (e.g. healthy cells).

Methods of determining cell surface presentation are well known in the art and include, but not limited to flow cytometry and immuno-cytochemistry.

According to specific embodiments, the binding domain is of an antibody and the target is an antigen.

According to other specific embodiments, the extracellular binding domain is not an antibody binding domain.

According to specific embodiments, the multi subunit protein module is devoid of an antibody binding domain.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to specific embodiments, the antibody is a whole or intact antibody.

According to specific embodiments, the antibody comprises an Fc domain.

According to specific embodiments, the antibody is an antibody fragment.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2, or antibody fragments comprising the Fc region of an antibody.

According to specific embodiments, the identity of the amino acid residues in the antibody that make up the variable region and/or the CDRs is determined by the method of Kabat et al. (See, e.g., Kabat et al., 1992, Sequences of Proteins of Immunological Interest, 5th ed., Public Health Service, NIH, Washington D.C.).

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

According to specific embodiments, the extracellular binding domain capable of binding the target comprises a scFv.

According to other specific embodiments, the extracellular binding domain is not an scFv.

According to specific embodiments, the multi subunit protein module is devoid of an scFv.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

It will be appreciated that for human therapy, humanized antibodies are preferably used.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to specific embodiments, the antibody is a human antibody.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147 (1): 86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

According to specific embodiments, the antibody binds an antigen overexpressed or only expressed by a pathologic cell e.g. cancerous cell.

Non-limiting examples for known cancer antigens include MAGE-AI, MAGE-A2, MAGE-A3, MAGE-A4, MAGE-AS, MAGE-A6, MAGE-A7, MAGE-AS, MAGE-A9, MAGE-AIO, MAGE-All, MAGE-A12, GAGE-I, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-1, SSX-2 (HOM-MEL-40), SSX-3, SSX-4, SSX-5, SCP-1 and XAGE, melanocyte differentiation antigens, p53, ras, CEA, MUCI, PMSA, PSA, tyrosinase, Melan-A, MART-I, gp100, gp75, alphaactinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-All, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, plSOerbB-3, c-met, nm-23Hl, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, C0-029, FGF-5, 0250, Ga733 (EpCAM), HTgp-175, M344, MA-50, MG7-Ag, MOV18, NB\170K, NYCO-I, RCASI, SDCCAG16, TA-90 (Mac-2 binding protein\cyclophilin C-associated protein), TAAL6, TAG72, TLP, TPS, tyrosinase related proteins, TRP-1, or TRP-2.

According to specific embodiments, the antibody binds EGFR, TRP-1, CD44, PDL-1, HER-2, MUC-1, MUC-16, CEA or EpCAM.

According to specific embodiments, the antibody binds EGFR.

According to specific embodiments, the antibody is an anti-EGFR comprising a variable heavy chain comprising SEQ ID NO: 41 and/or variable light chain comprising SEQ ID NO: 42.

According to a specific embodiment, the extracellular binding domain capable of binding the target comprises an anti-EGFR scFv comprising SEQ ID NO: 43.

According to other specific embodiments, the extracellular binding domain is of a ligand and the target is a receptor of the ligand. Non-limiting examples of such ligand-receptor pairs that can be used for targeting cancerous cells include a ligand of a tyrosine kinase receptor-tyrosine kinase receptor, EGF-EGFR, CD19 ligand-CD19, hyaluronic acid-CD44.

According to other specific embodiments, the extracellular binding domain is of a receptor and said target is a ligand of the receptor. Non-limiting examples of such receptor-ligand pairs that can be used for targeting cancerous cells include PD-1-PDL-1, CD137-CD137L, integrin alpha2beta1-E-Cadherin.

According to specific embodiments, the target is bound to the target cell. Thus, for example, the target may be an antibody or an Fc-fusion which is capable of binding an antigen expressed by a target cell.

Thus, according to specific embodiments, the extracellular binding domain is of an Fcγ receptor and the target is an Fc ligand bound to a target cell.

As used herein the phrase “extracellular binding domain of Fcγ receptor” refers to at least a fragment of an Fcγ receptor which comprises an extracellular domain capable of binding an Fc ligand.

As used herein, the term “Fc ligand” refers to an Fc domain such as of an antibody.

According to specific embodiments, the Fc ligand is an IgG Fc domain.

As used herein, the term “Fcγ receptor” refers to a cell surface receptor which exhibits binding specificity to the Fc domain of an IgG antibody. Examples of Fcγ receptors include, without limitation, CD64A, CD64B, CD64C, CD32A, CD32B, CD16A, and CD16B. The term “Fcγ receptor” also encompasses functional homologues (naturally occurring or synthetically/recombinantly produced) and/or Fc receptors comprising conservative and non-conservative amino acid substitutions, which exhibit the desired activity (i.e., capability of binding an IgG Fc binding domain).

According to specific embodiments, the Fcγ receptor is CD64.

As used herein, the term “CD64”, also known as FcγRI, refers to the polypeptide expression product of the FCGR1A, FCGR1B or FCGR1C gene (Gene ID 2209, 2210, 2211, respectively), and includes CD64A, CD64B and CD64C. Full length CD64 comprises an extracellular, transmembrane and an intracellular domain and is capable of at least binding an IgG (IgG1 and IgG3) Fc domain and recruiting an FcRγ. Methods of determining binding and recruitment of an FcRγ are well known in the art and are also described hereinabove and below.

According to specific embodiments, CD64 is human CD64. According to a specific embodiment, the CD64 protein refers to the human CD64A protein, such as provided in the following UniProt Number P12314.

According to a specific embodiment, the CD64 protein refers to the human CD64B protein, such as provided in the following UniProt Number Q92637.

According to a specific embodiment, the CD64 protein refers to the human CD64C protein, such as provided in the following GenBank Number XM_001133198.

The extracellular domain of full length CD64 comprises three immunoglobulin (Ig) domains referred to as D1-D3 from N to C.

According to specific embodiments, the extracellular binding domain of CD64 comprises all Ig domains D1-D3.

According to specific embodiments, the extracellular binding domain of CD64 comprises SEQ ID NO: 14, 44 or 64.

According to specific embodiments, the extracellular binding domain of CD64 consists of SEQ ID NO: 14, 44 or 64.

According to specific embodiments, the extracellular binding domain of CD64 comprises SEQ ID NO: 14 or 44.

According to specific embodiments, the extracellular binding domain of CD64 consists of SEQ ID NO: 14 or 44.

According to specific embodiments, the extracellular binding domain of CD64 comprises the two Ig domains D1-D2.

According to specific embodiments, the extracellular binding domain of CD64 comprises SEQ ID NO: 45 or 46.

According to specific embodiments, the extracellular binding domain of CD64 consists of SEQ ID NO: 45 or 46.

The term “extracellular binding domain of CD64” also encompasses functional homologues (naturally occurring or synthetically/recombinantly produced), which exhibit the desired activity (i.e., binding an IgG Fc domain). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 14, 44, 64, 45 or 46; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the extracellular binding domain of CD64 may comprise conservative and non-conservative amino acid substitutions.

According to specific embodiments, the at least one polypeptide comprising the extracellular binding domain comprises an amino acid sequence capable of recruiting other polypeptides comprising the amino acid sequence of FcRγ being part of the multi subunit protein module upon binding of the binding domain to the target.

Such recruitment will form a complex upon binding of the extracellular binding domain to the target, enabling transmission of an activating signal by the amino acid sequence of an FcRγ in an immune cell expressing the multi subunit protein module.

According to a specific embodiment, the recruitment refers to oligomerization e.g., trimerization of the at least three polypeptides being part of the multi subunit protein module.

According to specific embodiments, the amino acid sequence capable of recruiting the other polypeptides directly recruits the other polypeptide (i.e. without an intermediate polypeptide).

Such amino acid sequences are well known to the skilled in the art and include for example the transmembrane and/or the cytoplasmic domains of several Fc receptors such as, but not limited to CD64, CD16A, CD16B, FcεRIβ, FcαRI (CD89).

According to specific embodiments, the amino acid sequence capable of recruiting the other polypeptides is not of an Fcε receptor (FcεR).

Methods of determining recruitment of the polypeptides are well known in the art, and include, but are not limited to, enzymatic activity assays such as kinase activity assays, and expression of molecules involved in the signaling cascade using e.g. PCR, Western blot, immunoprecipitation and immunohistochemistry. Additionally or alternatively, determining recruitment of the polypeptides can be effected by evaluating cell activation or function by methods well known in the art such as, but not limited to proliferation assays such as CFSE staining, MTT, Alamar blue, BRDU and thymidine incorporation, cytotoxicity assays such as CFSE staining, chromium release, Calcin AM, and the like. Exemplary methods for determining recruitment are disclosed in e.g. in Kim, M. K., et al. (2003) Blood 101(11): 4479-4484; and Harrison, P. T., et al. (1995) Mol Membr Biol 12(4): 309-312, the contents of which are fully incorporated herein by reference.

According to specific embodiments, the amino acid sequence capable of recruiting the other polypeptides comprising the amino acid sequence of FcRγ comprises the transmembrane domain of an Fc receptor.

According to specific embodiments, the amino acid sequence capable of recruiting the other polypeptides comprising the amino acid sequence of FcRγ comprises the transmembrane domain of an Fcγ receptor.

According to specific embodiments, the amino acid sequence capable of recruiting the other polypeptides comprising the amino acid sequence of FcRγ comprises the transmembrane domain of CD64.

According to specific embodiments, the amino acid sequence capable of recruiting the other polypeptides comprising the amino acid sequence of FcRγ consists of the transmembrane domain of CD64.

According to specific embodiments, the amino acid sequence capable of recruiting the other polypeptides comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 16 or 47.

According to specific embodiments, the amino acid sequence capable of recruiting the other polypeptides comprises SEQ ID NO: 16 or 47.

According to specific embodiments, the amino acid sequence capable of recruiting the other polypeptides consists of SEQ ID NO: 16 or 47.

According to specific embodiments, both the extracellular binding domain and the amino acid sequence capable of recruiting the other polypeptides are of CD64.

Hence, according to specific embodiments, the at least one polypeptide which comprises the extracellular binding domain comprises the extracellular and the transmembrane domains of CD64.

According to specific embodiments, the at least one polypeptide which comprises the extracellular binding domain comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 18 or 48.

According to specific embodiments, the at least one polypeptide which comprises the extracellular binding domain comprises SEQ ID NO: 18 or 48.

According to specific embodiments, the at least one polypeptide which comprises the extracellular binding domain comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 19 or 49.

According to specific embodiments, the at least one polypeptide which comprises the extracellular binding domain comprises SEQ ID NO: 19 or 49.

According to specific embodiments, the at least one polypeptide which comprises the extracellular binding domain consists of SEQ ID NO: 19 or 49.

According to specific embodiments, at least two of the polypeptides in the multi subunit module are capable of forming a dimer.

The dimer may be a homodimer or a heterodimer.

According to specific embodiments, the dimer is a homodimer.

Thus, according to specific embodiments, at least two of the at least three cell membrane polypeptides comprising the amino acid sequence of FcRγ comprise a dimerizing moiety.

According to specific embodiments, the polypeptides devoid of the extracellular the binding domain are capable of forming a dimer.

Hence, according to specific embodiments, the polypeptides comprising the amino acid sequence of FcRγ and not comprising the binding domain comprise a dimerizing moiety.

As used herein, the term “dimerizing moiety” refers to an amino acid sequence capable of forming a polypeptide dimer. Such an amino acid may include for example an amino acid sequence comprising at least two cysteine residues enabling the formation of a disulfide bond between the thiol groups. Methods of determining dimerization are known in the art, including but not limited to immunoprecipitation, size exclusion chromatography, fast protein liquid chromatography (FPLC), multi-angle light scattering (SEC-MALS) analysis, SDS-PAGE analysis, nano-DSF, yeast two-hybrid system (e.g. RRS) and flow cytometry.

It will be appreciated that the dimerizing moiety of some embodiments of the invention is also capable of forming multimers (e.g., at least three).

Any known dimerizing moiety known in the art can be used with specific embodiments of the invention. A non-limiting example of such a dimerizing moiety which can be used with specific embodiments of the invention include an amino acid sequence of a transmembrane domain of FcRγ.

Thus, for example, according to specific embodiments, the dimerizing moiety comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 21 or 50.

According to specific embodiments, the dimerizing moiety comprises SEQ ID NO: 21 or to 50.

According to specific embodiments, the dimerizing moiety consists of SEQ ID NO: 21 or 50.

The multi subunit protein module of some embodiments of the invention comprises the following combination of elements: the binding domain is of an Fcγ receptor (e.g. CD64) and the target is an Fc ligand; the at least one polypeptide comprising said binding domain comprises a transmembrane domain of an Fcγ receptor (e.g. CD64); and the polypeptides comprising said amino acid sequence of FcRγ and not comprising said binding domain comprise as a dimerizing moiety an amino acid sequence of a transmembrane domain of said FcRγ.

Hence, according to an aspect of the present invention, there is provided a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises a transmembrane domain of CD64 and an extracellular binding domain of CD64 capable of binding an Fc ligand; and wherein said polypeptides comprising said amino acid sequence of FcRγ and not comprising said binding domain comprise as a dimerizing moiety an amino acid sequence of a transmembrane domain of said FcRγ, such that upon binding of said extracellular binding domain to said Fc ligand said activating signal is transmitted in an immune cell expressing said multi subunit protein module.

According to specific embodiments, at least one of the polypeptides in the multi subunit protein module comprises an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 23, 51 or 71.

According to specific embodiments, at least one of the polypeptides in the multi subunit protein module comprises SEQ ID NO: 23, 51 or 71.

According to specific embodiments, at least one of the at least three cell membrane polypeptides in the multi subunit protein module consists of SEQ ID NO: 23, 51 or 71.

According to specific embodiments, at least two of the polypeptides in the multi subunit protein module comprise an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 23, 51 or 71.

According to specific embodiments, at least two of the polypeptides in the multi subunit protein module comprise SEQ ID NO: 23, 51 or 71.

According to specific embodiments, at least two of the at least three cell membrane polypeptides in the multi subunit protein module consist of SEQ ID NO: 23,51 or 71.

A specific example of a multi subunit protein module encompassed by specific embodiments of the present invention comprises:
(i) A polypeptide dimer wherein each of the polypeptides in the dimer comprises an amino acid sequence of FcRγ capable of transmitting an activating signal (e.g. FcRγ intracellular domain) and a dimerizing moiety (e.g. FcRγ transmembrane domain); and
(ii) A polypeptide comprising an extracellular binding domain capable of binding a target presented by a target cell of an immune cell (e.g. CD64 extracellular domain, scFv), an amino acid sequence capable of recruiting the polypeptide dimer following binding of the binding domain the target (e.g. CD64 transmembrane 25 domain) and an amino acid sequence of FcRγ capable of transmitting an activating signal (e.g. FcRγ intracellular domain),
   such that upon binding of the extracellular binding domain to the target the activating signal is transmitted in an immune cell expressing the multi subunit protein module.

According to specific embodiments, the multi subunit protein module comprises:
(i) A polypeptide dimer wherein each of the polypeptides in the dimer comprises an FcRγ intracellular domain and a FcRγ transmembrane domain; and
(ii) A polypeptide comprising a CD64 extracellular domain, a CD64 transmembrane domain and an FcRγ intracellular domain.

A non-limiting example of such a multi subunit protein module is schematically demonstrated in FIG. 2B.

According to specific embodiments, each of the polypeptides in the polypeptide dimer of (i) comprises SEQ ID NO: 23, 51 or 71 and the polypeptide of (ii) comprises SEQ ID NO: 19 or 49.

According to specific embodiments, each of the polypeptides in the polypeptide dimer of (i) consists of SEQ ID NO: 23, 51 or 71 and the polypeptide of (ii) consists of SEQ ID NO: 19 or 49.

According to specific embodiments, the multi subunit protein module comprises:
(i) A polypeptide dimer wherein each of the polypeptides in the dimer comprises an FcRγ intracellular domain and a FcRγ transmembrane domain; and
(ii) A polypeptide comprising a scFv, a CD64 transmembrane domain and an FcRγ intracellular domain.

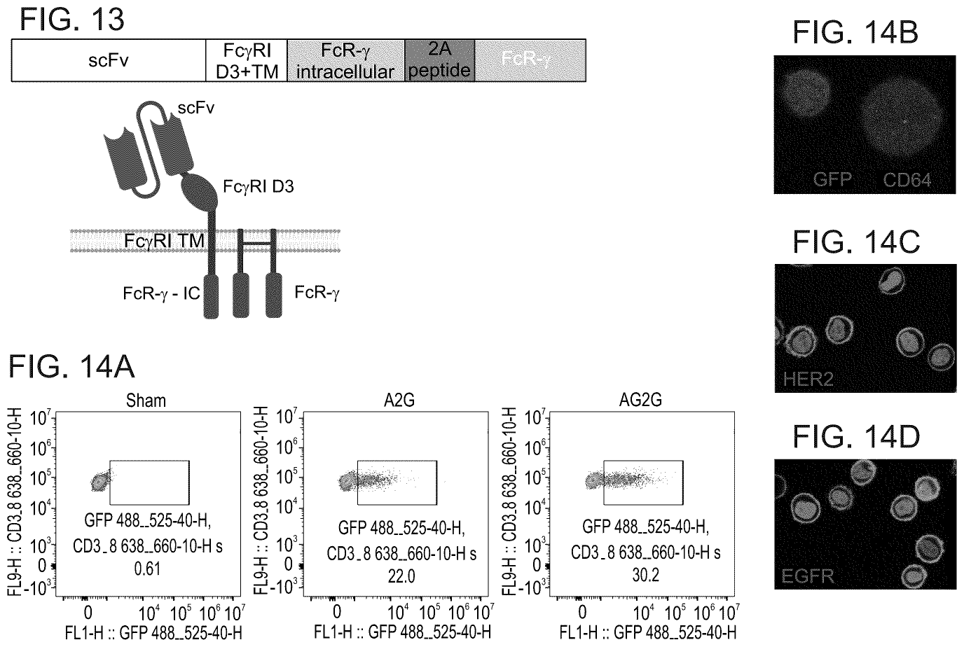

A non-limiting example of such a multi subunit protein module is schematically demonstrated in FIG. 13.

According to specific embodiments, each of the polypeptides in the polypeptide dimer of (i) comprises SEQ ID NO: 23, 51 or 71 and the polypeptide of (ii) comprises SEQ ID NO: 52.

According to specific embodiments, each of the polypeptides in the polypeptide dimer of (i) consists of SEQ ID NO: 23, 51 or 71 and the polypeptide of (ii) consists of SEQ ID NO: 52.

According to specific embodiments, any of the polypeptides disclosed herein can comprise a co-stimulatory signaling domain.

According to specific embodiments, the at least one polypeptide comprising the extracellular binding domain comprises a co-stimulatory signaling domain.

According to specific embodiments, the polypeptide devoid of the extracellular binding domain comprises a co-stimulatory signaling domain.

According to other specific embodiments, the polypeptides disclosed herein do not comprise a co-stimulatory signaling domain.

According to specific embodiments, the at least one polypeptide comprising the extracellular binding domain does not comprise a co-stimulatory signaling domain.

According to specific embodiments, the polypeptide devoid of the extracellular binding domain does not comprise a co-stimulatory signaling domain.

As used herein, the phrase “co-stimulatory signaling domain” refers to an amino acid sequence of a co-stimulatory molecule capable of transmitting a secondary stimulatory signal resulting in activation of an immune cell (e.g. T cell). Typically, a co-stimulatory signaling domain does not comprise an ITAM domain.

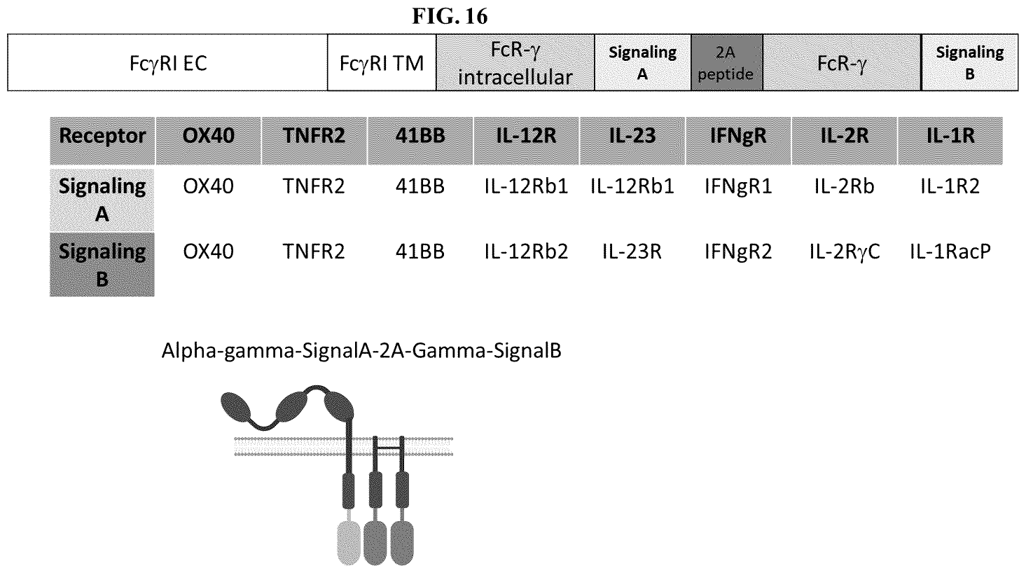

Any known co-stimulatory signaling domain can be used with specific embodiments of the present invention. Non-limiting examples of co-stimulatory signaling domains include 4-1BB, CD28, OX40, ICOS, CD27, GITR, HVEM, TIM1, LFA1(CD11a), CD2. Non-limiting schematic representations of costimulatory domains that can be added to the polypeptide of some embodiments of the invention are provided in FIG. 16.

According to specific embodiments, the co-stimulatory signaling domain is of 4-1BB and/or OX40.

Non-limiting examples of specific sequences of co-stimulatory signaling domains are provided in SEQ ID NOs: 25 (OX40), SEQ ID NO: 26 (4-1BB).

According to specific embodiments, any of the polypeptides disclosed herein can comprise a cytokine receptor signaling domain.

According to specific embodiments, the at least one polypeptide comprising the extracellular binding domain comprises a cytokine receptor signaling domain.

According to specific embodiments, the polypeptide devoid of the extracellular binding domain comprises cytokine receptor signaling domain.

According to other specific embodiments, the polypeptides disclosed herein do not comprise a cytokine receptor signaling domain.

According to specific embodiments, the at least one polypeptide comprising the extracellular binding domain does not comprise a cytokine receptor signaling domain.

According to specific embodiments, the polypeptide devoid of the extracellular binding domain does not comprise a cytokine receptor signaling domain.

As used herein, the phrase “cytokine receptor signaling domain” refers to an amino acid sequence of a cytokine receptor capable of transmitting a stimulatory signal resulting in activation of the T cell.

Any known cytokine receptor signaling domain can be used with specific embodiments of the present invention. Non-limiting examples of cytokine receptor signaling domains include IL2rg that is the IL2 receptor common gamma chain (e.g. such as provided e.g. in SEQ ID NO: 27), the Toll/IL1 receptor homology domain (TIR) that is the signaling domain of the myd88 receptor, TNF receptor intracellular domain (e.g. such as provided in SEQ ID NO: 28), IL12-Rb1 intracellular domain (e.g. such as provided in SEQ ID NO: 29), IL12-Rb1 intracellular domain (e.g. such as provided in SEQ ID NO: 30), IL23 receptor intracellular domain (e.g. such as provided in SEQ ID NO: 31), IFNγ receptor 1 intracellular domain (e.g. such as provided in SEQ ID NO: 32), IFNγ receptor 2 intracellular domain (e.g. such as provided in SEQ ID NO: 33), IL2Rb intracellular domain (e.g. such as provided in SEQ ID NO: 34), IL1 receptor intracellular domain (e.g. such as provided in SEQ ID NO: 35), IL1AcP receptor intracellular domain (e.g. such as provided in SEQ ID NO: 36).

Non-limiting schematic representations of cytokine receptor signaling domains that can be added to the polypeptide of some embodiments of the invention are provided in FIG. 16.

Any of the components comprised in a single polypeptide as described herein may be linked to each other directly of via a linker, each possibility represents a separate embodiment of the present invention.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et al., (2000), Protein Eng. 13(5):309-312, the entire contents of which are hereby incorporated by reference.

According to specific embodiments, the linker is a synthetic linker.

According to specific embodiments, the linker is a polypeptide.

Non-limiting examples of linkers that can be used include AS, GS, (GGGGS)ₙ (n=1-4) (SEQ ID NO: 53), GGGGSGGGG (SEQ ID NO: 54), (Gly)₈ (SEQ ID NO: 55), (Gly)₆ (SEQ ID NO: 56), (EAAAK)ₙ (n=1-3) (SEQ ID NO: 57), PAPAP (SEQ ID NO: 58).

According to specific embodiments, the linker is a GGGGSGGGGSGGGGS (SEQ ID NO: 59) linker.

According to a specific embodiment, the light chain and the heavy chain of the antibody (e.g. scFv) are linked via a linker, for example SEQ ID NO: 59.

According to specific embodiments, the linker comprises the extracellular Ig D3 domain of CD64 (e.g. SEQ ID NO: 60-61).

According to a specific embodiment, the antibody (e.g. scFv) is fused to the transmembrane domain of the polypeptide via a linker, for example SEQ ID NO: 60 or 61).

According to specific embodiments, the multi subunit protein module is produced by recombinant DNA technology.

Thus, according to an aspect of the present invention, there is provided at least one polynucleotide encoding the multi subunit protein module.

Hence, according to an aspect of the present invention, there is provided at least one polynucleotide encoding a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface of a target cell of an immune cell, such that upon binding of said extracellular binding domain to said target said activating signal is transmitted in an immune cell expressing said multi subunit protein module.

According to an additional or an alternative aspect of the present invention, there is provided at least one polynucleotide encoding a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises a transmembrane domain of CD64 and an extracellular binding domain of CD64 capable of binding an Fc ligand; and wherein said polypeptides comprising said amino acid sequence of FcRγ and not comprising said binding domain comprise as a dimerizing moiety an amino acid sequence of a transmembrane domain of said FcRγ, such that upon binding of said extracellular binding domain to said Fc ligand said activating signal is transmitted in an immune cell expressing said multi subunit protein module.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above). This term includes polynucleotides derived from naturally occurring nucleic acids molecules (e.g., RNA or DNA), synthetic polynucleotide molecules composed of naturally occurring bases, sugars, and covalent internucleoside linkages (e.g., backbone), as well as synthetic polynucleotides and/or oligonucleotides having non-naturally occurring portions, which function similarly to the respective naturally occurring portions.

Such a modified polynucleotide may comprise modification in either backbone, internucleoside linkages or bases. Modified polynucleotides may be preferred over native forms according to specific embodiments, because of desirable properties such as, for example, enhanced cellular uptake, enhanced affinity for nucleic acid target and increased stability in the presence of nucleases.

According to specific embodiments, the polynucleotide is a modified polynucleotide e.g. modified RNA.

According to specific embodiments, the at least one polynucleotide comprises a nucleic acid sequence encoding a first polypeptide comprising an extracellular and a transmembrane domain of CD64 and an intracellular domain of FcRγ; and a nucleic acid sequence encoding a second distinct polypeptide comprising an extracellular, a transmembrane and an intracellular domain of FcRγ.

According to specific embodiments, the at least three cell membrane polypeptides described herein are encoded by a single polynucleotide. Further description on expression of multiple polypeptides from a single polynucleotide is provided hereinbelow.

Thus, according to specific embodiments, the at least one polynucleotide is one polynucleotide.

According to other specific embodiments, several polynucleotides are used to encode the polypeptides of the multi subunit protein module.

According to specific embodiments, the at least three cell membrane polypeptides described herein are encoded by different polynucleotides.

According to a specific embodiment, the at least one polypeptide comprising the extracellular binding domain disclosed herein is encoded by a first polynucleotide and the polypeptides not comprising the extracellular binding domain disclosed herein are encoded by a second polynucleotide.

Thus, according to specific embodiments, the at least one polynucleotide is at least two polynucleotides.

According so specific embodiments, the at least one polynucleotide is two polynucleotides.

To express any of the disclosed polypeptides in cells, a polynucleotide sequence encoding the polypeptide(s) is preferably ligated into a nucleic acid construct suitable for cell expression. Such a nucleic acid construct includes at least one cis-acting regulatory element for directing expression of the nucleic acid sequence. Cis-acting regulatory sequences include those that direct constitutive expression of a nucleotide sequence as well as those that direct inducible expression of the nucleotide sequence only under certain conditions. Thus, for example, a promoter sequence for directing transcription of the polynucleotide sequence in the cell in a constitutive or inducible manner is included in the nucleic acid construct. In the case of mRNA, since gene expression from an RNA source does not require transcription, there is no need in a promoter sequence or the additional sequences involved in transcription described hereinbelow.

The nucleic acid construct (also referred to herein as an “expression vector”) of some embodiments of the invention includes additional sequences which render this vector suitable for replication and integration (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes or encodes a signal sequence for targeting the polypeptide to the cell surface. According to a specific embodiment, the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed, i.e. T cells. Examples of T cell specific promoters include lymphoid specific promoters [Calame et al., (1988) Adv. Immunol. 43:235-275]; in particular promoters of T-cell receptors [Winoto et al., (1989) EMBO J. 8:729-733].

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

A vector may also include a transcription terminator (e.g., from Bovine Growth Hormone (BGH) gene), an element allowing episomal replication and replication in prokaryotes (e.g. SV40 origin and ColEl or others known in the art) and/or elements to allow selection (e.g., ampicillin resistance gene and/or zeocin marker).

In order to assess the expression of a polypeptide or portions thereof, the expression vector can also contain either a selectable marker gene or a reporter gene or both to facilitate identification and selection of expressing cells from the population of cells sought to be transfected or transduced through viral vectors. Alternatively, the selectable marker may be carried on a separate polynucleotide and used in a co-transfection procedure. Both selectable markers and reporter genes may be flanked with appropriate regulatory sequences to enable expression in the host cells. Useful selectable markers include, for example, antibiotic-resistance genes, such as neo and the like. Reporter genes maybe be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the vector has been introduced into the host cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) or a self-cleavable peptide; and sequences for genomic integration of the promoter-chimeric polypeptide.

According to specific embodiments, the polypeptides of the multi subunit protein module described herein are expressed from distinct constructs.

According to other specific embodiments, the polypeptides of the multi subunit protein module described herein are expressed from a single construct in a multicistronic e.g. bicistronic manner. Such an expression can be achieved by method well known in the art such as, but not limited to, using internal ribosome entry site (IRES) sequence and/or a nucleic acid sequence encoding a self-cleavable peptide e.g. a 2A peptide (e.g. P2A, T2A, E2A).

Hence, according to specific embodiments, the polynucleotide or construct comprises a self-cleavable peptide (e.g. 2A skipping peptide, e.g. such as provided in SEQ ID NOs: 76-77).

According to specific embodiments, the polynucleotide or construct comprises an IRES sequence or a nucleic acid sequence encoding a self-cleavable peptide e.g. a 2A peptide between a nucleic acid sequence encoding a first polypeptide comprising an extracellular and a transmembrane domain of CD64 and an intracellular domain of FcRγ and a nucleic acid sequence encoding a second distinct polypeptide comprising an extracellular, a transmembrane and an intracellular domain of FcRγ.

A specific example of a polynucleotide encompassed by specific embodiments of the present invention comprises sequentially from 5′ to 3′ nucleic acid sequences encoding an extracellular domain of CD64, a transmembrane domain of CD64, an intracellular domain of FcRγ, a 2A skipping peptide, an extracellular domain of FcRγ, a transmembrane domain of FcRγ and an intracellular domain of FcRγ.

According to specific embodiments, the extracellular domain of CD64 the transmembrane domain of CD64 and the intracellular domain of FcRγ are translationally fused.

According to specific embodiments, the extracellular, transmembrane and intracellular domains of FcRγ are translationally fused.

Non-limiting examples of nucleic acid constructs encoding the multi subunit protein module of some embodiments of the invention are provided in SEQ ID NO: 4, 63 and 38.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Various methods of producing embodiments of the present invention may be employed. For example, a vector can be directly transduced into a cell, e.g., an immune cell e.g. a T cell or a NK cell. According to specific embodiments, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. According to specific embodiments, the vector is capable of expressing the polynucleotide in mammalian e.g. human T cells. According to specific embodiments, the vector is capable of expressing the polynucleotide in mammalian e.g. human NK cell.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the host cell. The ability to select suitable vectors for transforming T cells is well within the capabilities of the ordinary skilled artisan and as such no general description of selection consideration is provided herein.

The terms “infecting” and “transducing”, which are interchangeably used herein, refer to modification of cells through use of a viral vector.

Recombinant viral vectors are useful for expression of the polypeptides of some embodiments of the invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

The constructs described herein are suitable for introduction into cells of interest by various techniques. For example, an expression vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al, 2012, MOLECULAR CLONING: A LABORATORY MANUAL, volumes 1-4, Cold Spring Harbor Press, NY). An alternative method for the introduction of a polynucleotide into a host cell is lipofection, e.g., using Lipofectamine (Life Technologies). Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. In the case where a non-viral delivery system is utilized, an exemplary delivery vehicle is a liposome. The use of lipid formulations is contemplated for the introduction of the nucleic acids into a host cell (in vitro, ex vivo or in vivo). According to additional or alternative embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et ah, 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Methods of the embodiments may concern transfecting the cells with a DNA encoding the multi subunit protein module described herein and, in some cases, a transposase. Such methods of transfecting of cells may also employ highly efficient transfections methods, such as electroporation. For example, nucleic acids may be introduced into cells using a nucleofection apparatus. If such methods are employed, the transfection step preferably does not involve infecting or transducing the cells with virus, which can cause genotoxicity and/or lead to an immune response to cells containing viral sequences in a treated subject. Such methods may involve transfecting cells with an expression vector encoding the multi subunit protein module. A wide range of constructs and expression vectors for the same are known in the art and are further detailed herein. For example, in some embodiments, the expression vector is a DNA expression vector such as a plasmid, linear expression vector or an episome. In some embodiments, the vector comprises additional sequences, such as sequence that facilitate expression of the polynucleotide, such a promoter, enhancer, poly-A signal, and/or one or more introns. In some embodiments, the coding sequence is flanked by transposon sequences, such that the presence of a transposase allows the coding sequence to integrate into the genome of the transfected cell.

Some methods may require that cells are further transfected with a transposase that facilitates integration of a coding sequence into the genome of the transfected cells. In some embodiments, the transposase is provided as DNA expression vector. In others, the transposase is provided as an expressible RNA or a protein such that long-term expression of the transposase does not occur in the transgenic cells. For example, the transposase may be provided as an mRNA (e.g., an mRNA comprising a cap and poly-A tail). Any transposase system may be used in accordance with the embodiments. However, in some aspects, the transposase is salmonid-type Tcl-like transposase (SB). For example, the transposase can be the so called “Sleeping beauty” transposase, see e.g., U.S. Pat. No. 6,489,458, incorporated herein by reference. In certain aspects, the transposase is an engineered enzyme with increased enzymatic activity. Some specific examples of transposases include, without limitation, SB10, SB11 or SB100× transposase (see, e.g., Mates et al., 2009, incorporated herein by reference). For example, a method can involve electroporation of cells with a mRNA encoding a SB10, SB11 or SB100× transposase.

Regardless of the method used to introduce exogenous nucleic acids into a host cell or otherwise expose a cell to embodiments of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Modification of cells to express the multi subunit protein modules may be performed with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)].

Introduction of nucleic acids by viral infection offers several advantages over other methods such as lipofection and electroporation, since higher transduction efficiency can be obtained due to the infectious nature of viruses. For example, preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for targeting the polypeptide to the desired site in a cell. According to specific embodiments the signal sequence comprises a membrane trafficking sequence. Such sequences are known in the art. Non-limiting examples are provided in SEQ ID Nos: 72-73 and 74-75. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Specific embodiments of the present invention also contemplate immune cells expressing the multi subunit protein module described herein and method of generating same.

Thus, according to an aspect of the present invention, there is provided an immune cell genetically engineered to express the at least one polynucleotide.

According to an additional or an alternative aspect of the present invention, there is provided an immune cell expressing the at least one polynucleotide.

According to an additional or an alternative aspect of the present invention, there is provided an immune cell expressing at least one polynucleotide encoding a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface of a target cell of an immune cell, such that upon binding of said extracellular binding domain to said target said activating signal is transmitted in the immune cell.

According to an additional or an alternative aspect of the present invention, there is provided an immune cell expressing at least one polynucleotide encoding a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises a transmembrane domain of CD64 and an extracellular binding domain of CD64 capable of binding an Fc ligand; and wherein said polypeptides comprising said amino acid sequence of FcRγ and not comprising said binding domain comprise as a dimerizing moiety an amino acid sequence of a transmembrane domain of said FcRγ, such that upon binding of said extracellular binding domain to said Fc ligand said activating signal is transmitted in the immune cell.

According to an additional or an alternative aspect of the present invention, there is provided an immune cell expressing the multi subunit protein module.

According to an additional or an alternative aspect of the present invention, there is provided an immune cell expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface of a target cell of an immune cell, such that upon binding of said extracellular binding domain to said target said activating signal is transmitted in the immune cell.

According to an additional or an alternative aspect of the present invention, there is provided an immune cell expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises a transmembrane domain of CD64 and an extracellular binding domain of CD64 capable of binding an Fc ligand; and wherein said polypeptides comprising said amino acid sequence of FcRγ and not comprising said binding domain comprise as a dimerizing moiety an amino acid sequence of a transmembrane domain of said FcRγ, such that upon binding of said extracellular binding domain to said Fc ligand said activating signal is transmitted in the immune cell.

According to an additional or an alternative aspect of the present invention, there is provided a method of expressing a multi subunit protein module in an immune cell, the method comprising introducing into an immune cell the at least one polynucleotide, under conditions which allow expression of said multi subunit protein module.

Such conditions may be for example an appropriate temperature (e.g., 37° C.), atmosphere (e.g., air plus 5% CO₂), pH, light, medium, supplements and the like.

According to other specific embodiments, the introducing is effected in-vivo.

According to specific embodiments, the introducing is effected in-vitro or ex-vivo.

Methods of obtaining immune cells are well known in the art. Thus, for examples, PBMCs can be isolated by drawing whole blood from a subject and collection in a container containing an anti-coagulant (e.g. heparin or citrate); and apheresis. According to other specific embodiments, the immune cells are obtained from a tissue comprising cells associated with a pathology. Methods for obtaining a tissue sample from a subject are well known in the art and include e.g. biopsy, surgery or necropsy and preparing a single cell suspension thereof. Following, according to specific embodiments, at least one type of an immune cell is purified from the peripheral blood or from the single cell suspension. There are several methods and reagents known to those skilled in the art for purifying immune cells such as leukapheresis, sedimentation, density gradient centrifugation (e.g. ficoll), centrifugal elutriation, fractionation, chemical lysis of e.g. red blood cells (e.g. by ACK), selection of specific cell types using cell surface markers (using e.g. FACS sorter or magnetic cell separation techniques such as are commercially available e.g. from Invitrogen, Stemcell Technologies, Cellpro, Advanced Magnetics, or Miltenyi Biotec.), and depletion of specific cell types by methods such as eradication (e.g. killing) with specific antibodies or by affinity based purification based on negative selection (using e.g. magnetic cell separation techniques, FACS sorter and/or capture ELISA labeling). Such methods are described for example in THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 2000).

According to specific embodiments, the immune cell is a human cell.

According to specific embodiments, the immune cell is of a healthy subject.

According to specific embodiments, the immune cell is of a subject suffering from a pathology (e.g. cancer).

Non-limiting examples of immune cells that can be used with specific embodiments of the invention include T cells, NK cells, NKT cells, B cells, macrophages, monocytes, dendritic cells (DCs) and granulocytes.

According to specific embodiments, the immune cell is selected from the group consisting of T cells, NK cells and NKT cells.

According to specific embodiments, the immune cell does not express an endogenous FcRγ, as determined by flow cytometry or western blot.

According to specific embodiments, the immune cell is a T cell.

According to an additional or an alternative aspect of the present invention, there is provided a T cell expressing a polypeptide complex, wherein the polypeptide complex comprises at least a first polypeptide and a second polypeptide, wherein said first and second polypeptides are not translationally fused, wherein the first polypeptide comprises an amino acid sequence of an Fc receptor common γ chain (FcRγ), said amino acid sequence is capable of transmitting an activating signal and forming a homodimer; and said second polypeptide comprising:
(i) an extracellular ligand-binding domain of an Fcγ receptor (e.g. CD64) capable of binding an Fc ligand,
(ii) an amino acid FcRγ capable of transmitting an activating signal and
(iii) an amino acid sequence capable of recruiting said first polypeptide (e.g. the transmembrane domain of an Fc receptor),
   such that upon binding of said Fc ligand to said extracellular ligand-binding domain of said Fcγ receptor said activating signal is transmitted.

As used herein, the phrase “polypeptide complex” refers to a plurality of at least two distinct (i.e. not translationally fused) polypeptides which together have the activity of transmitting an activating signal in an immune cell expressing the polypeptide complex upon binding of the extracellular domain of the Fcγ receptor to its Fc ligand.

As used herein, the term “T cell” refers to a differentiated lymphocyte with a CD3+, T cell receptor (TCR)+ having either CD4+ or CD8+ phenotype.

According to specific embodiments, the T cell is an effector cell.

As used herein, the term “effector T cell” refers to a T cell that activates or directs other immune cells e.g. by producing cytokines or has a cytotoxic activity e.g., CD4+, Th1/Th2, CD8+ cytotoxic T lymphocyte.

According to specific embodiments, the T cell is a CD4+ T cell.

According to other specific embodiments, the T cell is a CD8+ T cell.

According to specific embodiments, the T cell is a αβ T cell.

According to specific embodiments, the T cell is a γδ T cell.

According to specific embodiments, the T cell is a naïve T cell.

According to specific embodiments, the T cell is a memory T cell. Non-limiting examples of memory T cells include effector memory CD4+ T cells with a CD3+/CD4+/CD45RA−/CCR7− phenotype, central memory CD4+ T cells with a CD3+/CD4+/CD45RA−/CCR7+ phenotype, effector memory CD8+ T cells with a CD3+/CD8+CD45RA−/CCR7− phenotype and central memory CD8+ T cells with a CD3+/CD8+CD45RA−/CCR7+ phenotype.

According to specific embodiments, the T cell is a proliferating cell.

As used herein, the phrase “proliferating cell” refers to a T cell that proliferated upon stimulation as defined by a cell proliferation assay, such as, but not limited to, CFSE staining, MTS, Alamar blue, BRDU, thymidine incorporation, and the like.

According to specific embodiments, the T cell is a proliferating CD4+ T cell.

According to specific embodiments, the T cell is a proliferating CD8+ T cell.

According to specific embodiments, the T cell is expressing a T cell receptor specific for a pathologic (diseased, e.g. cancerous) cell, i.e. recognizes an antigen presented in the context of MHC which is overexpressed or only expressed by a pathologic cell as compared to a non-pathologic cell. Non-limiting examples of cancer antigens are further described hereinabove.

According to specific embodiments, the T cell is endogenously expressing a T cell receptor specific for a pathologic cell (e.g. cancerous cell).

According to specific embodiments, the T cell is an engineered T cells transduced with a T cell receptor (TCR).

As used herein the phrase “transduced with a TCR” or “genetically engineered to express a TCR” refers to cloning of variable α- and β-chains from T cells with specificity against a desired antigen presented in the context of MHC. Methods of transducing with a TCR are known in the art and are disclosed e.g. in Nicholson et al. Adv Hematol. 2012; 2012:404081; Wang and Rivière Cancer Gene Ther. 2015 March;22(2):85-94); and Lamers et al, Cancer Gene Therapy (2002) 9, 613-623. According to specific embodiments, the TCR is specific for a pathologic cell.

According to specific embodiments, the T cell is an engineered T cells transduced with a chimeric antigen receptor (CAR).

As used herein, the phrase “transduced with a CAR” or “genetically engineered to express a CAR” refers to cloning of a nucleic acid sequence encoding a chimeric antigen receptor (CAR), wherein the CAR comprises an antigen recognition moiety and a T-cell activation moiety. A chimeric antigen receptor (CAR) is an artificially constructed hybrid protein or polypeptide containing an antigen binding domain of an antibody (e.g., a single chain variable fragment (scFv)) linked to T-cell signaling or T-cell activation domains. Method of transducing with a CAR are known in the art and are disclosed e.g. in Davila et al. Oncoimmunology. 2012 Dec. 1;1(9):1577-1583; Wang and Rivière Cancer Gene Ther. 2015 March;22(2):85-94); Maus et al. Blood. 2014 Apr. 24;123(17):2625-35; Porter DL The New England journal of medicine. 2011, 365(8):725-733; Jackson H J, Nat Rev Clin Oncol. 2016;13(6):370-383; and Globerson-Levin et al. Mol Ther. 2014;22(5):1029-1038. According to specific embodiments, the antigen recognition moiety is specific for a pathologic cell.

According to other specific embodiments, the T cell is not transduced (i.e. does not express) a CAR.

According to specific embodiments, the immune cells comprise NK cells.

As used herein the term “NK cells” refers to differentiated lymphocytes with a CD16+ CD56+ and/or CD57+ TCR-phenotype. NK are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

According to specific embodiments, the immune cells comprise NKT cells.

As used herein the term “NKT cells” refers to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance.

According to specific embodiments, the immune cells comprise B cells.

As used herein the term “B cells” refers to a lymphocyte with a B cell receptor (BCR)+, CD19+ and or B220+ phenotype. B cells are characterized by their ability to bind a specific antigen and elicit a humoral response.

According to specific embodiments, the immune cells comprise phagocytic cells.

As used herein, the term “phagocytic cells” refer to a cell that is capable of phagocytosis and include both professional and non-professional phagocytic cells. Methods of analyzing phagocytosis are well known in the art and include for examples killing assays, flow cytometry and/or microscopic evaluation (live cell imaging, fluorescence microscopy, confocal microscopy, electron microscopy). According to specific embodiments, the phagocytic cells are selected from the group consisting of monocytes, dendritic cells (DCs) and granulocytes.

According to specific embodiments, the immune cells comprise monocytes.

According to specific embodiments, the term “monocytes” refers to both circulating monocytes and to macrophages (also referred to as mononuclear phagocytes) present in a tissue.

According to specific embodiments, the monocytes comprise macrophages. Typically, cell surface phenotype of macrophages include CD14, CD40, CD11b, CD64, F4/80 (mice)/EMR1 (human), lysozyme M, MAC-1/MAC-3 and CD68.

According to specific embodiments, the monocytes comprise circulating monocytes. Typically, cell surface phenotypes of circulating monocytes include CD14 and CD16 (e.g. CD14++CD16−, CD14+CD16++, CD14++CD16+).

According to specific embodiments, the immune cells comprise DCs.

As used herein the term “dendritic cells (DCs)” refers to any member of a diverse population of morphologically similar cell types found in lymphoid or non-lymphoid tissues. DCs are a class of professional antigen presenting cells, and have a high capacity for sensitizing HLA-restricted T cells. DCs include, for example, plasmacytoid dendritic cells, myeloid dendritic cells (including immature and mature dendritic cells), Langerhans cells, interdigitating cells, follicular dendritic cells. Dendritic cells may be recognized by function, or by phenotype, particularly by cell surface phenotype. These cells are characterized by their distinctive morphology having veil-like projections on the cell surface, intermediate to high levels of surface HLA-class II expression and ability to present antigen to T cells, particularly to naive T cells (See Steinman R, et al., Ann. Rev. Immunol. 1991; 9:271-196.). Typically, cell surface phenotype of DCs include CD1a+, CD4+, CD86+, or HLA-DR. The term DCs encompasses both immature and mature DCs.

According to specific embodiments, the immune cells comprise granulocytes.

As used herein, the tern “granulocytes” refer to polymorphonuclear leukocytes characterized by the presence of granules in their cytoplasm.

According to specific embodiments, the granulocytes comprise neutrophils.

According to specific embodiments, the granulocytes comprise mast-cells.

According to specific embodiments, the immune cells can be freshly isolated, stored e.g., cryopreserved (i.e. frozen) at e.g. liquid nitrogen temperature at any stage for long periods of time (e.g., months, years) for future use; and cell lines.

Methods of cryopreservation are commonly known by one of ordinary skill in the art and are disclosed e.g. in International Patent Application Publication Nos. WO2007054160 and WO 2001039594 and US Patent Application Publication No. US20120149108.

According to specific embodiments, the immune cells can be stored in a cell bank or a depository or storage facility.

Consequently, specific embodiments of the present teachings further suggest the use of the immune cells (e.g. T cells) and the methods disclosed herein as, but not limited to, a source for adoptive immune cells therapies for diseases that can benefit from activating immune cells against pathologic cells e.g. a hyper-proliferative disease; a disease associated with immune suppression and infections.

Thus, according to an aspect of the present invention, the immune cells disclosed herein are for use in adoptive cell therapy.

The immune cells used according to specific embodiments of the present invention may be autologous or non-autologous; they can be syngeneic or non-syngeneic: allogeneic or xenogeneic to the subject; each possibility represents a separate embodiment of the present invention.

According to specific embodiments, the cells are autologous to said subject.

According to specific embodiments, the cells are non-autologous to said subject.

According to specific embodiments, the immune cells described herein are cultured, expanded and/or activated ex-vivo prior to administration to the subject.

Methods of culturing, expanding and activating immune cells are well known to the skilled in the art. For example, T cells may be activated ex vivo in the presence of one or more molecule such as, but not limited to, an anti-CD3 antibody, an anti-CD28 antibody, anti-CD3 and anti-CD28 coated beads (such as the CD3CD28 MACSiBeads obtained from Miltenyi Biotec), IL-2, phytohemoagglutinin, an antigen-loaded antigen presenting cell [APC, e.g. dendritic cell], a peptide loaded recombinant MHC.

Since the immune cells of specific embodiments of the present invention are activated upon binding of the extracellular binding domain to a target presented on a cell surface of a target cell of an immune cell, they may be used for, but not limited to, treating diseases associated with pathologic cells presenting the target on their cell surface.

Thus, according to an aspect of the present invention, there is provided a method of treating a disease associated with a pathologic cell in a subject in need thereof, the method comprising administering to the subject a therapeutically effective amount of the immune cell disclosed herein, wherein said pathologic cell presents said target on its cell surface, thereby treating the disease in the subject.

According to an additional or an alternative aspect of the present invention, there is provided the immune cell disclosed herein, for use in treating a disease associated with a pathologic cell in a subject in need thereof, wherein said pathologic cell presents said target on its cell surface.

According to an additional or an alternative aspect of the present invention, there is provided a method of treating a disease associated with a pathologic cell in a subject in need thereof, the method comprising administering to the subject a therapeutically effective amount of T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface said pathologic cell, such that upon binding of said extracellular binding domain to said target said activating signal is transmitted in the T cells, thereby treating the disease in the subject.

According to an additional or an alternative aspect of the present invention, there is provided a therapeutically effective amount of T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain capable of binding a target that is presented on a cell surface a pathologic cell, such that upon binding of said extracellular binding domain to said target said activating signal is transmitted in the T cells, for use in treating a disease associated with said pathologic cell in a subject in need thereof.

Since the immune cells of specific embodiments of the present invention are activated upon binding of the extracellular binding domain to a target bound to a cell surface of a pathologic cell, they may be used for, but not limited to, treating diseases associated with pathologic cells in combination with a therapeutic composition comprising the target which is directed for binding the pathologic cells, i.e. binds an antigen overexpressed or only expressed by a pathologic (e.g. cancerous) cell as compared to a non-pathologic cell. For examples, when the extracellular binding domain is of CD64 and the target is an Fc ligand; the therapeutic composition comprises an Fc domain (e.g. antibody) which is directed for binding an antigen overexpressed or only expressed by the pathologic cells.

Thus, according to specific embodiments, the subject is treated with a therapeutic composition comprising said target, said therapeutic composition being specific for said pathologic cell.

According to specific embodiments, the therapeutic composition comprising the Fc domain is specific for a pathologic cell.

According to specific embodiments, the method comprises administering to said subject a therapeutically effective amount of a therapeutic composition comprising said target, said therapeutic composition being specific for said pathologic cell.

According to an additional or an alternative aspect of the present invention, there is provided a method of treating a disease associated with a pathologic cell in a subject in need thereof, the method comprising administering to the subject a therapeutically effective amount of the immune cell disclosed herein, and a therapeutic composition comprising said target, said therapeutic composition being specific for said pathologic cell, thereby treating the disease in the subject.

According to an additional or an alternative aspect of the present invention, there is provided the immune cell disclosed herein and a therapeutic composition comprising said target, for use in treating a disease associated with a pathologic cell in a subject in need thereof, wherein said therapeutic composition being specific for said pathologic cell.

As used herein, the term “subject” or “subject in need thereof” includes mammals, preferably human beings at any age or gender. The subject may be healthy or showing preliminary signs of a pathology, e.g. cancer. This term also encompasses individuals who are at risk to develop the pathology.

As used herein the term “treating” refers to curing, reversing, attenuating, alleviating, minimizing, suppressing or halting the deleterious effects of a disease or disorder (e.g. cancer). Those of skill in the art will understand that various methodologies and assays can be used to assess the development of a pathology, and similarly, various methodologies and assays may be used to assess the reduction, remission or regression of a pathology (e.g. a malignancy), as discussed below.

As used herein, the term “preventing” refers to keeping a disease, disorder or condition from occurring in a subject who may be at risk for the disease, but has not yet been diagnosed as having the disease.

As used herein the phrase, “disease associated with a pathologic cell” means that pathologic cells drive onset and/or progression of the disease.

According to specific embodiments, the disease can benefit from activating the immune cells of the subject.

As used herein the phrase “a disease that can benefit from activating immune cells” refers to diseases in which the subject's immune response activity may be sufficient to at least ameliorate symptoms of the disease or delay onset of symptoms, however for any reason the activity of the subject's immune response in doing so is less than optimal.

Non-limiting examples of diseases treated by some embodiments of the invention include hyper-proliferative diseases, diseases associated with immune suppression, immunosuppression caused by medication (e.g. mTOR inhibitors, calcineurin inhibitor, steroids) and infections.

According to specific embodiments, the disease comprises an infection.

As used herein, the term “infection” or “infectious disease” refers to a disease induced by a pathogen. Specific examples of pathogens include, viral pathogens, bacterial pathogens e.g., intracellular mycobacterial pathogens (such as, for example, Mycobacterium tuberculosis), intracellular bacterial pathogens (such as, for example, Listeria monocytogenes), or intracellular protozoan pathogens (such as, for example, Leishmania and Trypanosoma).

Specific types of viral pathogens causing infectious diseases include, but are not limited to, retroviruses, circoviruses, parvoviruses, papovaviruses, adenoviruses, herpesviruses, iridoviruses, poxviruses, hepadnaviruses, picornaviruses, caliciviruses, togaviruses, flaviviruses, reoviruses, orthomyxoviruses, paramyxoviruses, rhabdoviruses, bunyaviruses, coronaviruses, arenaviruses, and filoviruses.

Specific examples of viral infections which may be treated according to specific embodiments of the present invention include, but are not limited to, human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, rabies, etc.

According to specific embodiments, the disease comprises a hyper-proliferative disease.

According to specific embodiments, the hyper-proliferative disease comprises sclerosis, fibrosis, Idiopathic pulmonary fibrosis, psoriasis, systemic sclerosis/scleroderma, primary biliary cholangitis, primary sclerosing cholangitis, liver fibrosis, prevention of radiation-induced pulmonary fibrosis, myelofibrosis or retroperitoneal fibrosis.

According to other specific embodiments, the hyper-proliferative disease comprises cancer.

Thus, according to specific embodiments the pathologic cell is a cancerous cell.

Cancers which may be treated by some embodiments of the invention can be any solid or non-solid tumor (including liquid cancer), cancer metastasis and/or a pre-cancer.

According to specific embodiments, the cancer is a malignant cancer.

Examples of cancer include but are not limited to, carcinoma, blastoma, sarcoma and lymphoma. More particular examples of such cancers include, but are not limited to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the cancer is a pre-malignant cancer.

Pre-cancers are well characterized and known in the art (refer, for example, to Berman J J. and Henson D E., 2003. Classifying the pre-cancers: a metadata approach. BMC Med Inform Decis Mak. 3:8). Examples of pre-cancers include, but are not limited to, acquired small pre-cancers, acquired large lesions with nuclear atypia, precursor lesions occurring with inherited hyperplastic syndromes that progress to cancer, and acquired diffuse hyperplasias and diffuse metaplasias. Non-limiting examples of small pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia).

Non-limiting examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Non-limiting examples of precursor lesions occurring with inherited hyperplastic syndromes that progress to cancer include atypical mole syndrome, C cell adenomatosis and MEA. Non-limiting examples of acquired diffuse hyperplasias and diffuse metaplasias include Paget's disease of bone and ulcerative colitis.

Examples of solid tumors that can be treated by the instant methods include tumors and/or metastasis (wherever located) other than lymphatic cancer, for example brain and other central nervous system tumors (including but not limited to tumors of the meninges, brain, spinal cord, cranial nerves and other parts of central nervous system, e.g. glioblastomas or medulla blastomas); head and/or neck cancer; breast tumors; circulatory system tumors (including but not limited to heart, mediastinum and pleura, and other intrathoracic organs, vascular tumors and tumor-associated vascular tissue); excretory system tumors (including but not limited to tumors of kidney, renal pelvis, ureter, bladder, other and unspecified urinary organs); gastrointestinal tract tumors (including but not limited to tumors of oesophagus, stomach, small intestine, colon, colorectal, rectosigmoid junction, rectum, anus and anal canal, tumors involving the liver and intrahepatic bile ducts, gall bladder, other and unspecified parts of biliary tract, pancreas, other and digestive organs); oral cavity tumors (including but not limited to tumors of lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx); reproductive system tumors (including but not limited to tumors of vulva, vagina, Cervix uteri, Corpus uteri, uterus, ovary, and other sites associated with female genital organs, placenta, penis, prostate, testis, and other sites associated with male genital organs); respiratory tract tumors (including but not limited to tumors of nasal cavity and middle ear, accessory sinuses, larynx, trachea, bronchus and lung, e.g. small cell lung cancer or non-small cell lung cancer); skeletal system tumors (including but not limited to tumors of bone and articular cartilage of limbs, bone articular cartilage and other sites); skin tumors (including but not limited to malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites.

In some examples, the solid tumor treated by the methods of the instant disclosure is pancreatic cancer, bladder cancer, colon cancer, liver cancer, colorectal cancer (colon cancer or rectal cancer), breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, skin cancer, ocular tumor, choriocarcinoma (tumor of the placenta), sarcoma or soft tissue cancer.

In some examples, the solid tumor to be treated by the methods of the instant disclosure is selected bladder cancer, bone cancer, breast cancer, cervical cancer, CNS cancer, colon cancer, ocular tumor, renal cancer, liver cancer, lung cancer, pancreatic cancer, choriocarcinoma (tumor of the placenta), prostate cancer, sarcoma, skin cancer, soft tissue cancer or gastric cancer.

In some examples, the solid tumor treated by the methods of the instant disclosure is breast cancer. Non limiting examples of breast cancer that can be treated by the instant methods include ductal carcinoma in situ (DCIS or intraductal carcinoma), lobular carcinoma in situ (LCIS), invasive (or infiltrating) ductal carcinoma, invasive (or infiltrating) lobular carcinoma, inflammatory breast cancer, triple-negative breast cancer, paget disease of the nipple, phyllodes tumor (phylloides tumor or cystosarcoma phyllodes), angiosarcoma, adenoid cystic (or adenocystic) carcinoma, low-grade adenosquamous carcinoma, medullary carcinoma, papillary carcinoma, tubular carcinoma, metaplastic carcinoma, micropapillary carcinoma, and mixed carcinoma.

In some examples, the solid tumor treated by the methods of the instant disclosure is bone cancer. Non limiting examples of bone cancer that can be treated by the instant methods include osteosarcoma, chondrosarcoma, the Ewing Sarcoma Family of Tumors (ESFTs).

In some examples, the solid tumor treated by the methods of the instant disclosure is skin cancer. Non limiting examples of skin cancer that can be treated by the instant methods include melanoma, basal cell skin cancer, and squamous cell skin cancer.

In some examples, the solid tumor treated by the methods of the instant disclosure is ocular tumor. Non limiting examples of ocular tumor that can be treated by the methods of the instant disclosure include ocular tumor is choroidal nevus, choroidal melanoma, choroidal metastasis, choroidal hemangioma, choroidal osteoma, iris melanoma, uveal melanoma, intraocular lymphoma, melanocytoma, metastasis retinal capillary hemangiomas, congenital hypertrophy of the RPE, RPE adenoma or retinoblastoma. In some embodiments, cancers that are treated are liquid cancers. Examples of liquid cancers that can be treated by the methods provided herein include, but are not limited to, leukemias, myelomas, and liquid lymphomas. In specific embodiments, liquid cancers that can be treated in accordance with the methods described include, but are not limited to, liquid lymphomas, leukemias, and myelomas. Exemplary liquid lymphomas and leukemias that can be treated in accordance with the methods described include, but are not limited to, acute myelogenous leukemia (AML), myelodysplastic syndromes (MDS), chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin lymphomas (nodular sclerosis, mixed cellularity, lymphocyte-rich, lymphocyte depleted or not depleted), and nodular lymphocyte-predominant Hodgkin lymphoma. In one aspect, myelodysplastic syndromes (MDS) is a heterogenous group of clonal, hematopoietic stem cell disorders characterized by distinct morphological bone marrow changes, abnormal blood counts, common cytogenetic abnormalities, and recurrent mutations. MDS can predominantly occur in the elderly. Treatment of MDS can be based on risk stratification, with the International Prognostic Scoring System (IPSS) or revised IPSS (IPSS-R) being the most common classification systems. Low-risk MDS patients can receive supportive care or hematopoietic growth factors. A subset of patients with 5q deletions can be treated with lenalidomide. High-risk patients can be treated with hypomethylating agents (e.g., azacitidine, decitabine), intensive chemotherapy, and/or allogeneic stem cell transplantation. In some cases, MDS patients can be transformed to AML. Some MDS patients can develop progressive bone marrow failure and/or die of complications related to neutropenia (e.g., infection) or thrombocytopenia (e.g., bleeding). Initial management of MDS can be based on risk stratification. The newer IPSS-R can place patients into 5 categories: very good, good, intermediate, high, and very-high risk groups. Patients in the very good, good, and select intermediate-risk patients can be categorized as “low-risk,” whereas high, very high, and certain intermediate-risk patients can be categorized as the “high-risk” group. Azacitidine (5′-azacytidine) and decitabine (5′-aza-2′-deoxycytidine), which both are cytosine analogues, can lead to inhibition of DNA-methyltransferases (DNMTs) and can act as hypomethylating agents.

In another aspect, acute myeloid leukemia (AML) is characterized by the proliferation and accumulation of myeloid cells with accompanying hematopoietic failure. AML can be caused by chemical exposure, prior chemotherapy and radiation, or other environmental toxins.

According to specific embodiments, the cancer is selected from the group consisting of melanoma, lymphoma, colon cancer, lung cancer, breast cancer and pancreatic cancer.

According to specific embodiments, the cancer is melanoma or lymphoma.

According to specific embodiments, the cancer or the cancerous cell expresses a marker selected from the group consisting of PDL-1, E-Cadherin, CD19, MUC1, TRP-1 and TRP-2.

According to specific embodiments, the cancer or the cancerous cell expresses PDL-1.

As mentioned, according to specific embodiments, the immune cells are administered to the subject in combination with a therapeutic composition comprising the target.

Thus, for examples, when the target is an Fc ligand, the therapeutic composition comprises an Fc domain. Therapeutic compositions comprising Fc domains specific for pathologic cells are well known in the art and include, but not limited to, Fc-fusion proteins and antibodies.

According to specific embodiments, the Fc domain is of an IgG antibody.

According to specific embodiments, the therapeutic composition is an Fc-fusion protein.

As used herein the term, “Fc-fusion protein” refers to a molecule comprising an amino acid sequence capable of binding a pathologic cell (i.e. an antigen overexpressed or only expressed on a pathologic cell) combined with an Fc domain of an antibody.

Selection of the Fc-fusion protein used is well within the capability of those skilled in the art, and depends on the type of the disease and the antigens expressed by the pathologic cells associated with the pathology.

Non-limiting examples of Fc-fusion proteins that can be used with specific embodiments are disclosed in Weidle et al. Cancer Genomics and Proteomics (2012) 9(6): 357-372; and Sioud et al. Molecular Therapy-Methods & Clinical Development (2015) 2, 15043, the contents of which is fully incorporated herein by reference.

According to specific embodiments, the therapeutic composition is an antibody.

Selection of the therapeutic antibody used is well within the capability of those skilled in the art, and depends on the type of the disease and the antigens expressed by the pathologic cells associated with the pathology.

According to specific embodiments, the therapeutic antibody binds an antigen overexpressed or only expressed by tumor cells. Non-limiting examples of tumor antigens are further described hereinabove.

According to specific embodiments, the antibody is an IgG antibody (e.g. IgG1, IgG2, IgG3, IgG4).

According to a specific embodiment the antibody isotype is IgG1 or IgG3.

According to specific embodiments, the therapeutic antibody is an anti-TRP-1 or an anti-CD44 antibody.

According to some embodiments of the invention, the therapeutic antibody is selected from the group consisting of Atezolizumab, Avelumab, Alemtuzumab, Cetuximab, Panitumumab, Nimotuzumab, Rituximab, Gatipotuzumab (previously known as PankoMab-GEX®), Trastuzumab, Alemtuzumab, Bevacizumab, Ofatumumab, Pertuzumab, ofatumumab, obinutuzumab and IVIG.

According to specific embodiments, the therapeutic antibody is selected from the group consisting of Atezolizumab, Rituximab, Cetuximab, Gatipotuzumab and IVIG.

According to specific embodiments, the therapeutic antibody is an anti-PDL-1.

According to specific embodiments, the cancerous cell expresses PDL-1 and the therapeutic antibody is an anti-PDL-1.

According to specific embodiments, the antibody is Atezolizumab.

According to an aspect of the present invention, there is provided a method of treating a disease associated with a pathologic cell in a subject in need thereof, the method comprising administering to the subject a therapeutically effective amount of:
(i) an antibody specific for said pathologic cell; and
(ii) T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain of CD64 capable of binding an Fc ligand, such that upon binding of said extracellular binding domain to said Fc ligand said activating signal is transmitted in said T cell, thereby treating the disease in the subject.

According to an additional or an alternative aspect of the present invention, there is provided a therapeutically effective amount of:
(i) an antibody specific for a pathologic cell; and
(ii) T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain of CD64 capable of binding an Fc ligand, such that upon binding of said extracellular binding domain to said Fc ligand said activating signal is transmitted in said T cell,
   for use in treating a disease associated with said pathologic cell in a subject in need thereof.

According to an additional or an alternative aspect of the present invention, there is provided a method of increasing the killing capacity of an antibody against a pathologic cell in a subject in need thereof, the method comprising administering to the subject a therapeutically effective amount of:
(i) an antibody specific for the pathologic cell; and
(ii) T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain of CD64 capable of binding an Fc ligand, such that upon binding of said extracellular binding domain to said Fc ligand said activating signal is transmitted in said T cell,
   thereby increasing the killing capacity of the antibody against the pathologic cell.

The administration of the immune cells and the administration of the therapeutic composition can be effected in the same route or in separate routes.

The administration of the immune cells may be following or concomitant with the therapeutic composition comprising the target.

According to specific embodiments, the immune cells disclosed herein are administered to the subject following treatment with the therapeutic composition comprising the target.

According to other specific embodiments, the immune cells disclosed herein are administered to the subject concomitantly with the therapeutic composition comprising the target.

Multiple rounds of administration of the immune cells and multiple doses of the therapeutic composition comprising the target can be administered. Thus, according to specific embodiments, administering the immune cells disclosed herein is effected following at least one administration of the therapeutic composition comprising the target. According to specific embodiments, administering the cells disclosed herein is effected in a sequential order with the treatment with the therapeutic composition comprising the target.

According to specific embodiments, the multi subunit protein module is more efficient (e.g. at least 1.5 fold, at least 2 fold, at least 3 fold, at least 4 fold) in activating an immune cell (e.g. T cell) expressing the module following binding to the target as compared to an immune cell of the same type expressing only a single polypeptide comprising the extracellular binding domain capable of binding the target and the amino acid sequence of FcRγ capable of transmitting the activating signal; or a peptide complex comprising a first polypeptide comprising the amino acid sequence of FcRγ capable of transmitting the activating signal; and a second polypeptide comprising the extracellular binding domain capable of binding the target which is devoid of the amino acid sequence of FcRγ capable of transmitting the activating signal.

According to specific embodiments, this increase in efficiency may be manifested by reduced ratio of effector immune cells:target cells (e.g. less than 4:1, less than 5:1, less than 6:1, less than 7:1, less than 8:1).

According to specific embodiments, this increase in efficiency may be manifested by reduced number of immune cells infused to the subject.

According to specific embodiments, the immune cells and the therapeutic compositions comprising the target disclosed herein can be administered to a subject in combination with other established or experimental therapeutic regimen to treat a disease associated with pathologic cells (e.g. cancer) including, but not limited to analgesics, chemotherapeutic agents, radiotherapeutic agents, cytotoxic therapies (conditioning), hormonal therapy and other treatment regimens (e.g., surgery) which are well known in the art.

According to specific embodiments, the method of treatment involves first using pre-conditioning for cell therapy. Thus, the method of some embodiments comprises administering a pre-conditioning agent prior to administering the immune cells (e.g. T cells). For example, pre-conditioning patients prior to T cell therapies typically improves the efficacy of the T cell therapy by reducing the number of endogenous lymphocytes and increasing the serum level of homeostatic cytokines and/or pro-immune factors present in the patient. This creates a more optimal microenvironment for the transplanted T cells to proliferate once administered to the patient, and reduces the number of endogenous lymphocytes. Non-limiting examples of pre-conditioning agents include cyclophosphamide and/or fludrabine.

The immune cells disclosed herein and/or the therapeutic compositions disclosed herein can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the immune cells and/or the therapeutic composition accountable for the biological effect.

According to specific embodiments, the immune cells are the active ingredient in the formulation.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, intradermal, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to a specific embodiment, the immune cells disclosed herein or the pharmaceutical composition comprising same is administered via an IV route.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Alternative embodiments include depots providing sustained release or prolonged duration of activity of the active ingredient in the subject, as are well known in the art.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another aspect of the present invention there is provided an article of manufacture comprising a packaging material packaging the immune cells disclosed herein and a therapeutic composition comprising said target.

According to specific embodiments, the therapeutic composition is specific for a pathologic cell.

According to specific embodiments, the immune cell is a T cell and the therapeutic composition is an antibody.

According to an aspect of the present invention there is provided an article of manufacture comprising a packaging material packaging an antibody and T cells expressing a multi subunit protein module comprising at least three cell membrane polypeptides each comprising an amino acid sequence of an Fc receptor common γ chain (FcRγ), said amino acid sequence is capable of transmitting an activating signal; wherein at least one but not all of said at least three polypeptides comprises an extracellular binding domain of an Fcγ receptor (e.g. CD64) capable of binding an Fc ligand, such that upon binding of said extracellular binding domain to said Fc ligand said activating signal is transmitted in said T cell. According to specific embodiments, the article of manufacture is identified for the treatment of a disease associated with a pathologic cell (e.g. cancer).

According to specific embodiments, the immune cells (e.g. T cell) disclosed herein; and the therapeutic composition comprising the target (e.g. antibody) are packaged in separate containers.

According to specific embodiments, the immune cells (e.g. T cell) disclosed herein; and the therapeutic composition comprising the target (e.g. antibody) are packaged in a co-formulation.

According to specific embodiments, the article of manufacture further comprises a pre-conditioning agent. Further description and non-limiting exemplary agents are further provide hereinabove.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

Each patent, publication, and non-patent literature cited in the application is hereby incorporated by reference in its entirety as if each was incorporated by reference individually.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice—Wild-type (WT) C57BL/6 mice or BALB/c mice were obtained from Envigo (Jerusalem, Israel), and from Jackson Laboratories (Bar-Harbor, ME, USA). All mice were housed in an American Association for the Accreditation of Laboratory Animal Care-accredited animal facility and maintained in specific pathogen-free conditions. Male and female 8-12 weeks old mice were used in all experiments. All animal experiments were approved by the Tel-Aviv University or the Stanford University Institutional Animal Care and Use Committees.

Cell lines—B16F10 cells (CRL-6475, ATCC), YUMM1.7 (CRL-3362, ATCC), HEK-293FT (Thermo Fisher Scientific, Waltham, MA), A20 cells (Nahmad et al. bioRxiv 2020.02.28.970822; doi: wwwdoi(dot)org/10.1101/2020.02.28.970822) Jurkat cells (Aqaue et al. Cancer Res. 2019 Aug. 1;79(15):3862-3876)), 293GP (Burns J C et al. PNAS USA. 1993 Sep. 1;90(17):8033-7) and HT-29 (human colon adenocarcinoma, HTB-38, ATCC), were cultured in DMEM (GIBCO) supplemented with 10% heat-inactivated FBS (Biological Industries, Israel), 2 mM L-glutamine, and 100 μg/mL penicillin/streptomycin (GIBCO) under standard conditions. Cells were routinely tested for mycoplasma using EZ-PCR Mycoplasma Test Kit (Biological Industries, Israel) according to manufacturer's instructions. B16F10 and YUMM1.7 cells were infected by lentivirus containing pLVX-H2B-tdTomato, and were sorted by FACS (BD FACSAria™ III, BD Biosciences, Franklin Lakes, NJ) for the high-expressing tdTomato population. NK-92 cells (CRL-2407, ATCC) were cultured in RPMI supplemented with 200 U of human IL-2 (Peprotech).

Lentiviral infection—HEK-293FT cells were transfected with pLVX plasmids containing H2B-tdTomato under EF1 promoter together with psPAX2 (Addgene plasmid #12260) and pCMV-VSV-G (Addgene plasmid #8454). Media-containing viruses were collected following 24 and 48 hours. For infection, B16F10 or HT-29 cells were incubated with viruses and 100 μg/mL polybrene (Sigma Aldrich, Merck, Israel) for 30 minutes followed by 30 minutes centrifugation before medium was replaced. Following three days, cells that expressed tdTomato were sorted by FACSAriaII.

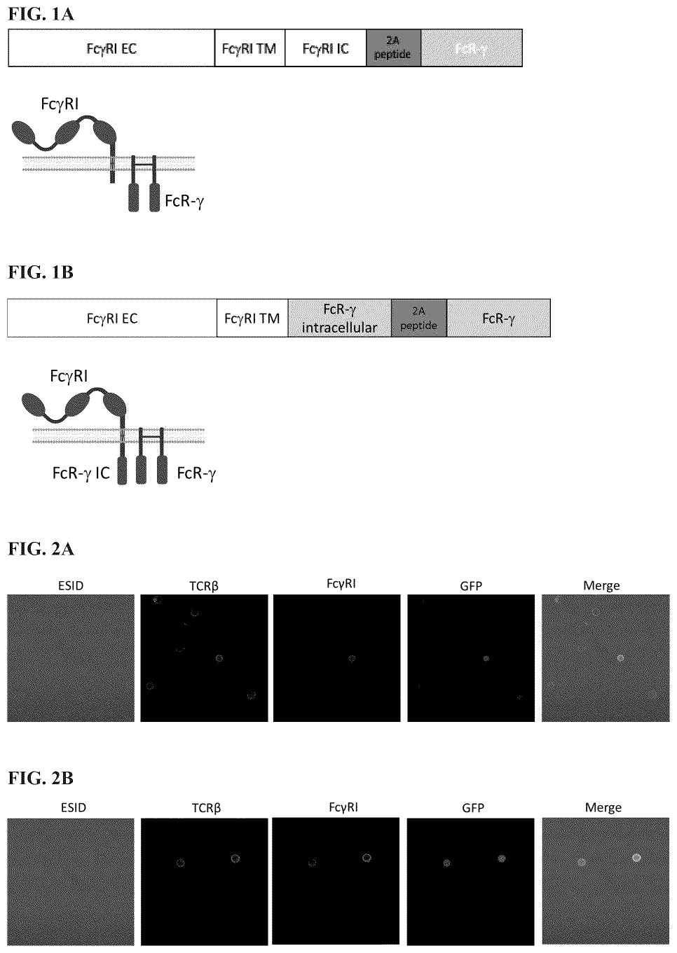

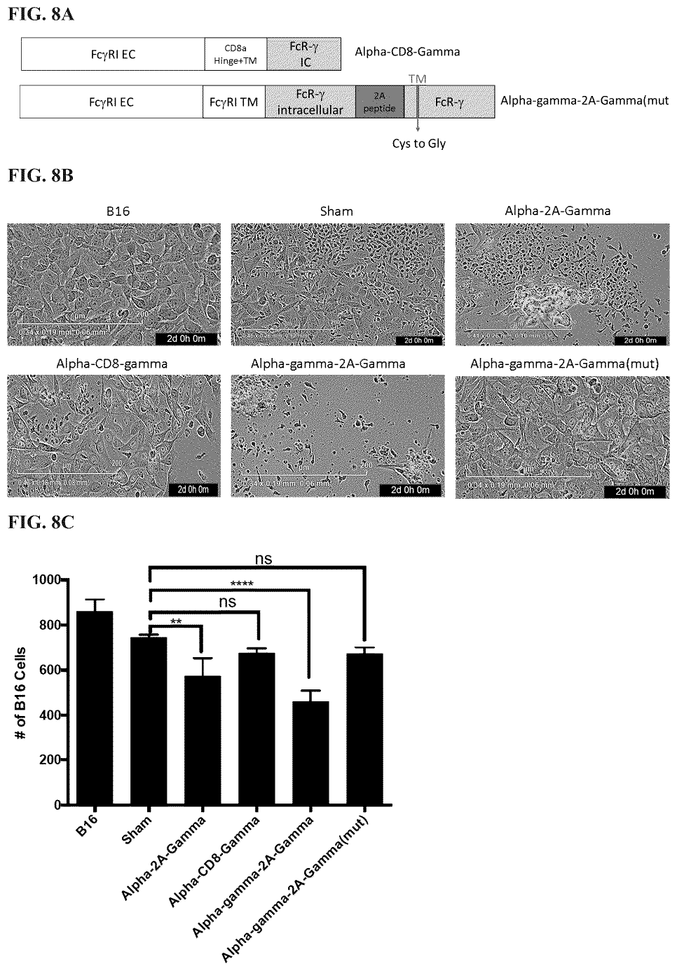

Constructs design—Several constructs were generated to express FcγRI and FcRγ (see FIGS. 1A, 1B and 8A), SEQ ID Nos: 1-8). Specifically, inserts of the fusion sequences were synthesized by GeneART (Thermo Fisher Scientific) into pMK vectors and were further cloned into pMIGII using EcoRI/XhoI sites upstream to IRES-GFP sequences or into pMSVG.1 vector cut with NcoI/NotI. Clones were verified by pBABE5′ and IRES-Rev primers sequencing (HyLabs Israel). Histone H2B sequence was amplified with AATAACACTAGTGCCACCATGCCTGAACCGGCAAAAT (SEQ ID NO: 9) and AACAACCCCGGGACTTGTCGTCATCGTCTTTGT (SEQ ID NO: 10) primers and cloned into pLVX vector (Clontech) containing EF1 promoter into SpeI/XmaI sites in frame with tdTomato. Sequence was verified by MSC\v forward and tdTomato reverse primers (HyLabs Israel). Extracellular and transmembrane and 7 residues of intracellular region of human HER2 or EGFR were cloned into pLVX vector using SpeI/NotI or EoRI/NotI respectively) sites.

Retroviral infection—Mouse total CD3⁺ T cells or specific CD4⁺ or CD8⁺ T cells were isolated from mouse blood and infected with the above constructs as follows: Platinum E cells were plated on 10 cm culture plates and co-transfected with 2:1 molar ratio of pMIGII⁴⁵ and PCL-Eco plasmids using Polyplus jetPRIME® reagent (Polyplus transfections). Following 24 hours, media was replaced with complete DMEM supplemented with 0.075% Sodium Bicarbonate. Media-containing viruses were collected after 24 hours and 48 hours and centrifuged for 1 hour at 100,000 g. Pellet was resuspended gently in 1 mL media and let to recover overnight at 4° C. Prior to infection, splenic CD4⁺ T cells or splenic CD8⁺ T cells were incubated on plate pre-coated with anti-CD3 (0.5 μg/mL) in T cell media containing high-dose IL-2 (1,000 IU/ml). Next, 0.3 mL of concentrated retroviruses were added to every 2×10⁶ CD4⁺ or CD8⁺ T cells with 10 μg/mL polybrene. Cells were incubated for 30 minutes in 37° C., 5% CO₂ and centrifuged at 37° C. 1,200 rpm for 1 hour. Following, 80% of medium was replaced and T cells were cultured for additional three days in T cell media containing high-dose IL-2. For infection of human cells (PBMC, NK-92 or γδ-T cells), 1.2×10⁶ 293GP cells were cultured in PLL-coated 6 well plate, transfected using JetOptimus (Polyplus transfections) with 1.4 μg RD114 envelope plasmid and 2 μg pMIGII or pMSVG.1 plasmid. 48 hours later, supernatants were collected, filtered with 0.45 μM PVDF filter, and 1.5×10⁵ cells were infected in 24 well suspension plate coated with Retronectin (Takara).

Primary human cells—Human PBMCs were collected from healthy donor, separated using Ficoll Histopaque-1077 HYBRI-MAX (Sigma aldrich), activated for 48 hours with Ultra-LEAF™ Purified anti-human CD3 Antibody (OKT3, Biolegend), together with 1000 U IL-2 (Peprotech), followed by retrovirus infection. For γδ T cells, human PBMCs from healthy donor were collected and cultured for 8 days with Zoledronate (5 uM, Novartis) and IL-2 (500 U, Peprotech), separated using EasySep™ Human Gamma/Delta T Cell Isolation Kit (StemCell technologies), and transduced with a retrovirus vector as described.

Confocal microscopy—Infected CD3⁺ or CD4⁺ and CD8⁺ T cells were plated on glass-bottom confocal plates and stained using anti-CD3 (clone 17A2, BioLegend), anti-TCRβ (H57-597), anti-FcRI (X54-5/7.1, BioLegend). A20 cells were stained using anti-B220 antibody (RA-3-6B2, BioLegend). Human primary cells were stained with anti-human CD3 (HIT3a, BioLegend), Alexa Fluor® 647 anti-human TCR γ/δ (B1, BioLegend) APC-anti-human CD64 (10.1, BioLegend) APC anti-human CD56 (5.1H11, BioLegend). HT-29 were stained with Alexa Fluor® 488 anti-human CD340 (erbB2/HER-2, 24D2, BioLegend), Alexa Fluor® 488 anti-human EGFR Antibody (AY13, BioLegend). Images were collected using a Zeiss LSM800 confocal laser scanning microscope and analyzed using ZEN software (Carl Zeiss Microscopy).

Killing assay—IncuCyte imager killing assays were conducted by culturing 10⁴ H2B-tdTomato B16F10 or YUMM1.7-H2B target cells in 96 wells plate with uninfected or infected CD4⁺ or CD8+ T cells at several effector: target ratios. Following 2 hours, where indicated, cells were added with 15 μg anti-TRP-1 (TA99, BioXcell) or anti-CD44 (IM7, BioLegend) antibodies in 200 μl medium. For human cells killing assays, 10⁴ HT-29 were cultured in 96 well plate; 24 hours later human effector cells were added in the specified ratios, with or without 60 μg/ml Trastuzumab (HY-P9907, MedChemExpress) or Cetuximab (HY-P9905, MedChemExpress). Cells were imaged by incuCyte S3 imager (Sartorius) for 24-60 hours. Images were then used to calculate numbers of target cells by incuCyte software. Human T cells activity was also evaluated by measuring the TNFα cytokine levels using Human TNF-alpha DuoSet ELISA (DY210, R&D Systems) and IFNγ levels using Human IFN-gamma DuoSet ELISA (DY285, R&D Systems). A20 killing was evaluated following 24 hours co-culture of A20 cells in a u-shaped 96 wells plate with infected CD8+ T cells isolated from BALB/c splenocytes, with or without 10 μg anti-CD20 antibody (18B12, Absolute antibody). Killing of A20 cells was determined by APC-Annexin V staining kit (BioLegend) in flow cytometer.

Flow cytometry—Purified T cells were analyzed using flow cytometry (CytoFLEX, Beckman Coulter, Lakeview Indianapolis, IA) for expressed GFP or by staining with Alexa Fluor® 647 anti-mouse CD64 (FcγRI) Antibody (X54-5/7.1, BioLegend), Brilliant Violet 421 or APC anti-human CD64 Antibody (10.1, BioLegend), Alexa Fluor® 488-anti-human CD3 (HIT3a, BioLegend). A20 cells were stained by PE-anti CD45R/B220 antibody (RA-3-6B2, BioLegend). Datasets were analyzed using FlowJo software (Tree Star).

In vivo tumor models—2×10⁵ B16F10 cells suspended in 50 μL DMEM were injected sub-cutaneously (s.c.) to C57BL/6 mice above the right flank and the size of growing tumors was measured twice a week using calipers. When tumors reached 120 mm², mice were sacrificed for ethical considerations. Animal were injected intravenously (i.v.) with 2×10⁶ uninfected (sham) or retroviral infected with pMIGII-alpha-gamma-2A-Gamma (SEQ ID NO: 4) CD3+ T cells, with or without 300 μg anti-TRP1 antibody (clone TA99) s.c. Treatment was applied at days 7, 11 and 14 post tumor injection.

Statistical analyses—Each experiment was performed three times. Each experimental group consisted of at least three mice. Significance of results was determined using the nonparametric one-way ANOVA, when multiple groups are analyzed, or nonparametric Student's t-test.

### Example 1

### Exogenous Expression of FcRγ and FcγRI Fused to the Intracellular Domain of FcRγ in CD4+ and CD8+ T Cells Induces Effective Tumor Cell Lysis

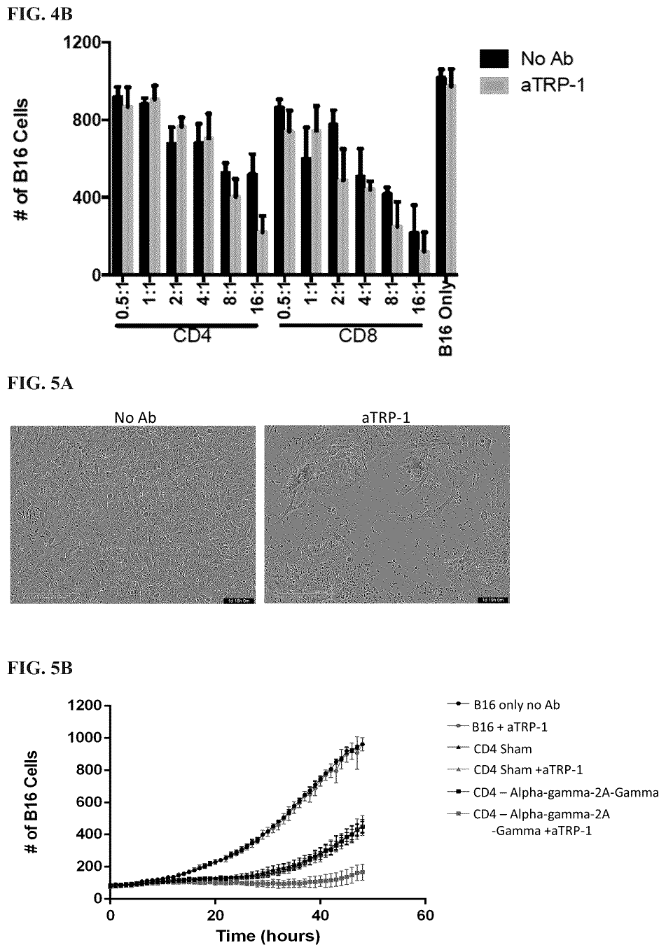

The following constructs were cloned: the extracellular, transmembrane and intracellular domains of FcγRI, T2A skipping peptide and FcRγ (SEQ ID NOs: 1-2), denoted herein as Alpha-2A-gamma (FIG. 1A); the extracellular and transmembrane domains of FcγRI fused to the intracellular domain of FcRγ, T2A skipping peptide and FcRγ (SEQ ID NOs: 3-4), denoted herein as Alpha-gamma-2A-gamma (FIG. 1B). These plasmids were packed into retrovirus, and used to infect CD4⁺ and CD8⁺ T cells. To ensure membrane localization of these constructs, cells were stained for TCR and CD3, and for FcRI alpha (FcγRI, CD64). Confocal analysis indicated that in both constructs FcRI was uniformly localized on T cell membrane (FIGS. 2A-B). As the therapeutic strategy of some embodiments is based on re-infusing autologous T cells expressing the above constructs along with a tumor-binding antibody, the killing activity of the transduced T cells was evaluated in-vitro on B16 cells which express histone H2B-tdTomato with or without aTRP-1 antibody, which binds the melanoma antigen gp75 on B16 cells. (FIGS. 4A-B and 5A-C).

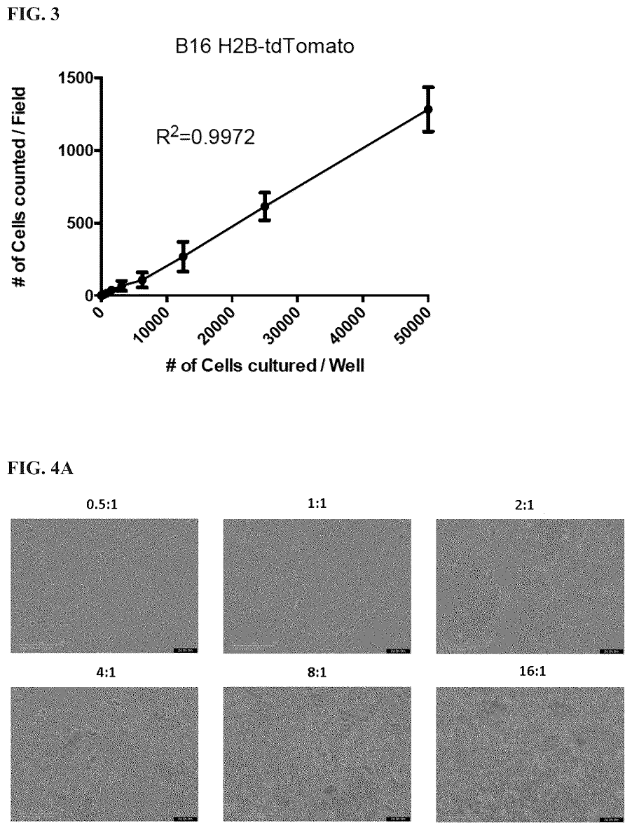

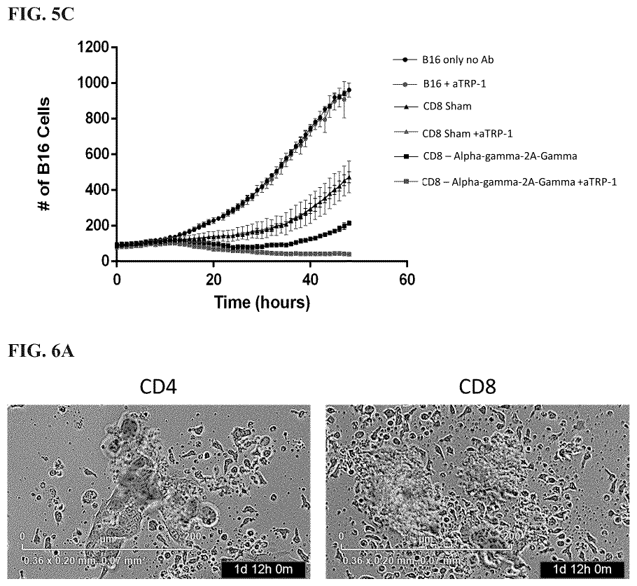

Initially, B16-H2B-tdTomato were cultured in serial concentrations ranging from 24 cells to 50,000 per well, imaged in incuCyte and counted by incuCyte analysis tool which detect and count the red fluorescent nuclei in a field captured by the camera. The graph in FIG. 3 shows a direct correlation between the amount of cell cultured and numbers of cell counted in a field. Consequently, the incuCyte imaging system was used to evaluate killing of B16-H2B-tdTomato by anti-TRP-1 antibody and T cells expressing Alpha-2A-gamma or Alpha-gamma-2A-gamma cultured in different effector:target ratios, ranging from 0.5:1 to 16:1. Representative images (FIG. 4A) and target cells numbers (FIG. 4B) after 48 hours show that both CD8⁺ and CD4⁺ T cells infected with Alpha-2A-gamma killed the tumor cells when the effector:target ratio is 8 to 1, or higher. The Alpha-gamma-2A-Gamma construct was much more potent compared to Alph-2A-Gamma, and induced target cells killing in effector:target ratio of 2:1 (FIGS. 5A-C).

Taken together, these results demonstrate that concomitant signaling through FcγRI and the FcRγ signaling chain can exert killing capacities in conventional CD4⁺ and CD8+ T cells whenever the target cells are coated with antibodies. In addition, a comparison shows the advantage of the Alpha-gamma-2A-Gamma construct over the Alpha-2A-gamma construct.

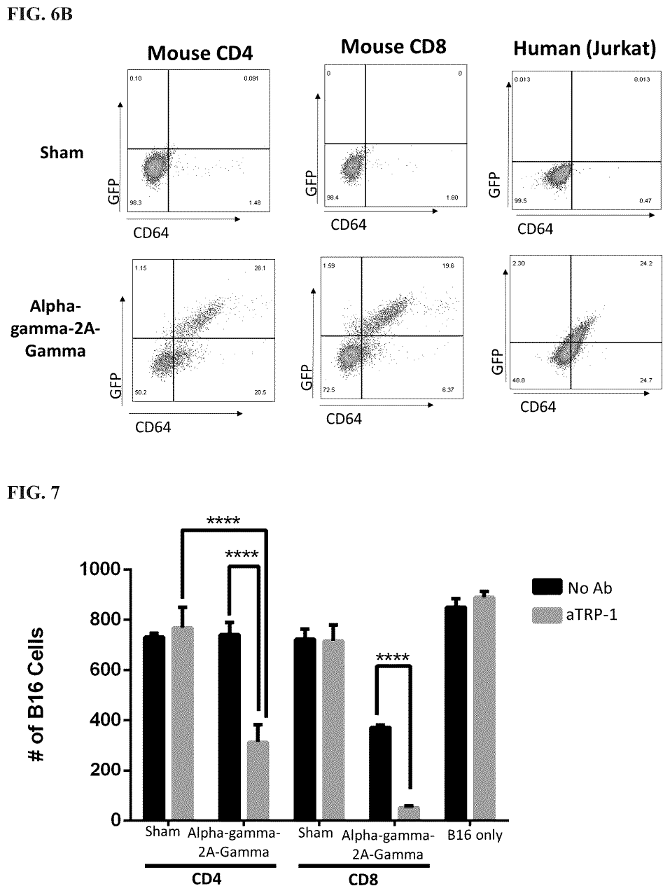

Since the B16-H2B-tdTomato target cells can be distinguished by their red nucleus and the infected cells express GFP originated from the pMIGII vector backbone, their activity could be monitored in real time. Indeed, the majority of tumor cell killing was mediated by the infected T cells as manifested by their GFP expression (FIG. 6A). In order to assess the exogenous expression of FcRI in the CD4+ and CD8+ T cells of mouse origin and in human Jurkat T cells infected with the Alpha-gamma-2A-Gamma, the cells were analyzed for GFP and FcRI (CD64) expression by flow cytometry (FIG. 6B). The analysis indicated that both mouse CD4+ and CD8+ cells and human Jurkat T cell line, have positive correlation between GFP levels and CD64 expression.

Following, the killing activity of Alpha-gamma-2A-Gamma infected CD4+ and CD8+ T cells, with or without anti-TRP-1 antibody was further evaluated in-vitro by counting the B16 tumor cell numbers following 60 hours of co-culturing (FIG. 7). The numbers of B16-H2B-tdTomato target cells counted by the incuCyte imager are shown in comparison to a control of B16 H2B-tdTomato target cells cultured alone. Most importantly, both CD4+ and CD8+ T cells exerted increased killing capacities compared to non-infected T cells.

In addition, the present inventors tested whether FcRI alpha fused to gamma chain can promote tumor cell killing, comparable to that observed when separating the gamma chain from the FcRI alpha chain (e.g. Alpha-2A-gamma and Alpha-gamma-2A-gamma). Therefore, FcRI alpha chain extracellular D1-D2 domains were cloned fused to CD8 hinge and transmembrane domains of CD8a together with signaling intracellular gamma (denoted herein as Alpha-CD8-gamma, SEQ ID NOs: 5-6) as illustrated in FIG. 8A. Furthermore, the present inventors tested if the gamma chain must form a dimer in Alpha-gamma-2A-Gamma by using a sequence with mutated cysteine residues in the transmembrane domain of the Gamma chain, denoted herein as Alpha-gamma-2A-Gamma(mut) (SEQ ID NOs: 7-8, FIG. 8A). Following, CD8+ T cell were infected with the Alpha-2A-Gamma construct (SEQ ID NO: 2), the Alpha-CD8-gamma construct (SEQ ID NO: 6), Alpha-gamma-2A-Gamma construct (SEQ ID NO: 4) or the Alpha-gamma-2A-Gamma(mut) construct (SEQ ID NO: 8) and were co-cultured with B16-H2B-tdTomato with or without an anti-TRP-1 antibody. Representative images are shown of B16-H2B-tdTomato target cells treated with an anti-TRP-1 antibody either alone or following co-culturing of T-cells that are unmodified CD8+ T-cells, CD8+ T cells transduced with Alpha-2A-Gamma, Alpha-CD8-Gamma, Alpha-gamma-2A-Gamma, or Alpha-gamma-2A-Gamma (mut) (FIG. 8B). Target cell numbers were estimated (FIG. 8C). While Alpha-2A-Gamma and Alpha-gamma-2A-Gamma infected T cells in combination with an anti-TRP-1 antibody induced tumor cell killing, Alpha-CD8-gamma and Alpha-gamma-2A-Gamma(mut) infected T cells were almost inert (FIGS. 8B-C). In addition, tumor cell killing was the most pronounced upon combined treatment with anti-TRP-1 and Alpha-gamma-2A-Gamma infected T cells. FIG. 8C indicates a value of around 750 B16 cells when a sham was used. In contrast, Alpha-2A-gamma resulted in a greater reduction, with around 575 B16 cells (FIG. 8C). Alpha-gamma-2A-Gamma had the greatest tumor cell killing, with around 460 B16 cells (FIG. 8C).

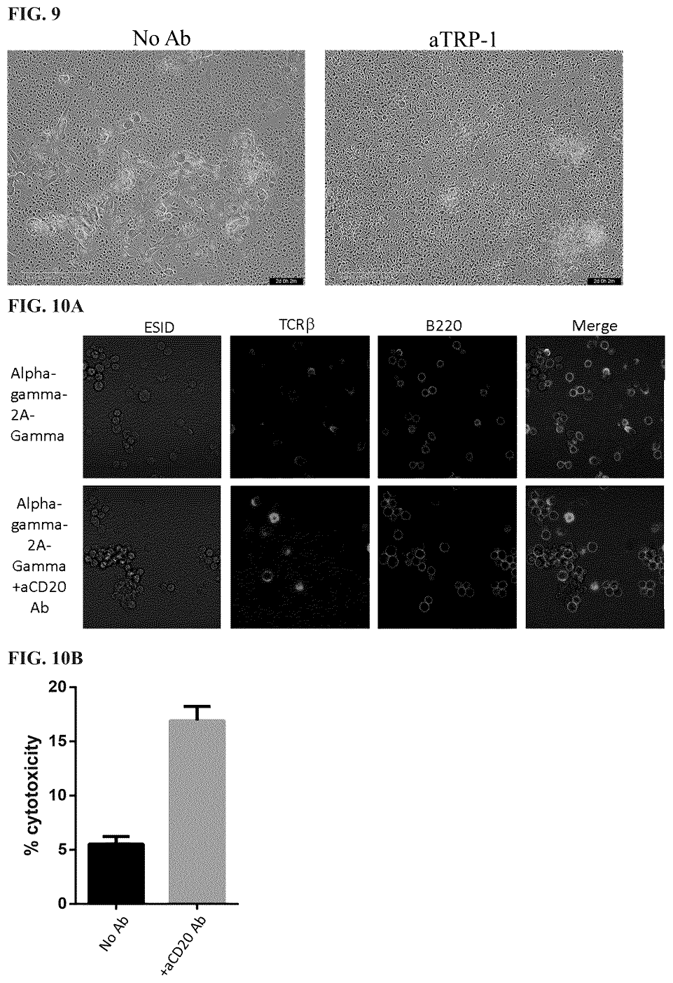

Subsequently, the ability of Alpha-gamma-2A-Gamma infected cells to kill another melanoma cell lines was evaluated. To this end, Yumm1.7 melanoma cells expressing H2B-tdTomato were incubated for 48 hours with CD8+ T cells expressing Alpha-gamma-2A-Gamma with or without an anti-TRP-1 antibody. Consistent with the results with the B16 cells, significant killing of Yumm1.7 tumor cells was observed upon addition of anti-TRP-1 (FIG. 9).

Following, the ability of Alpha-gamma-2A-Gamma infected cells to kill tumor cell types other than melanoma was evaluated. To this end, A20 B-cell lymphoma originated from BALB/C mice was incubated overnight with syngeneic CD8+ T cells infected with the Alpha-gamma-2A-Gamma construct and with an anti-CD20 antibody. FIG. 10A shows representative confocal microscopy of the co-culture where T cells are stained for TCRP and A20 lymphoma cells are stained with B220. Analysis of A20 cell death by Annexin-V staining by flow cytometry showed about 20% killing upon culturing with Alpha-gamma-2A-Gamma infected T cells and anti-CD20 antibody (FIG. 10B).

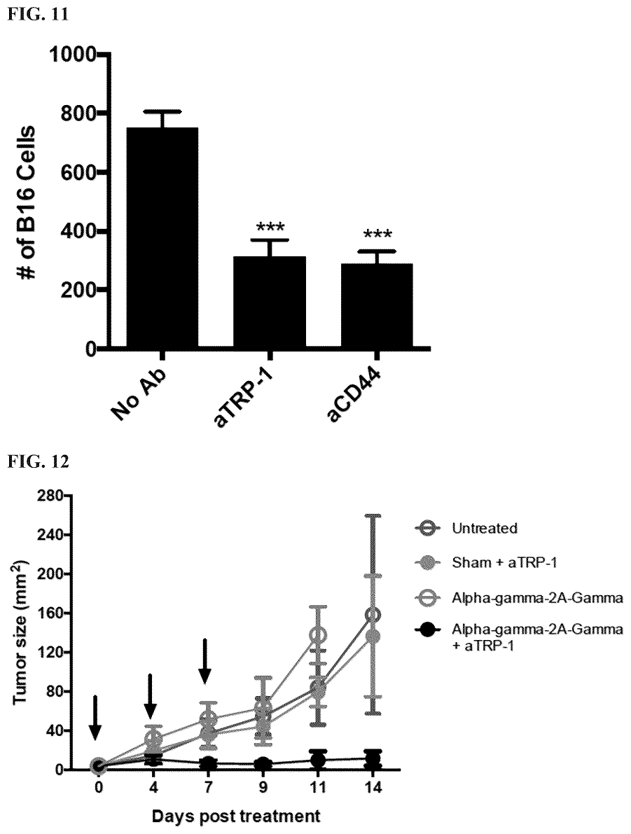

In addition, the ability of tumor-binding antibody other than anti-TRP-1 to mediate tumor cell killing by the Alpha-gamma-2A-Gamma infected T cells was evaluated. To this end, CD8+ T cells expressing the Alpha-gamma-2A-gamma construct were cultured with B16 cells along with an anti-CD44 antibody which binds the tumor cells. Indeed, anti-CD44 induced tumor cell killing, comparable to that induced by an anti-TRP1 antibody (FIG. 11).

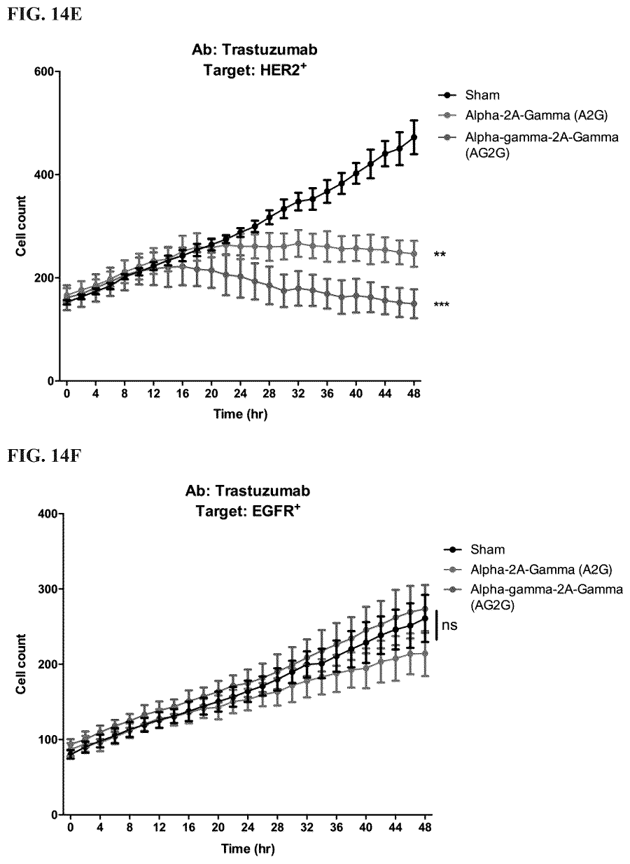

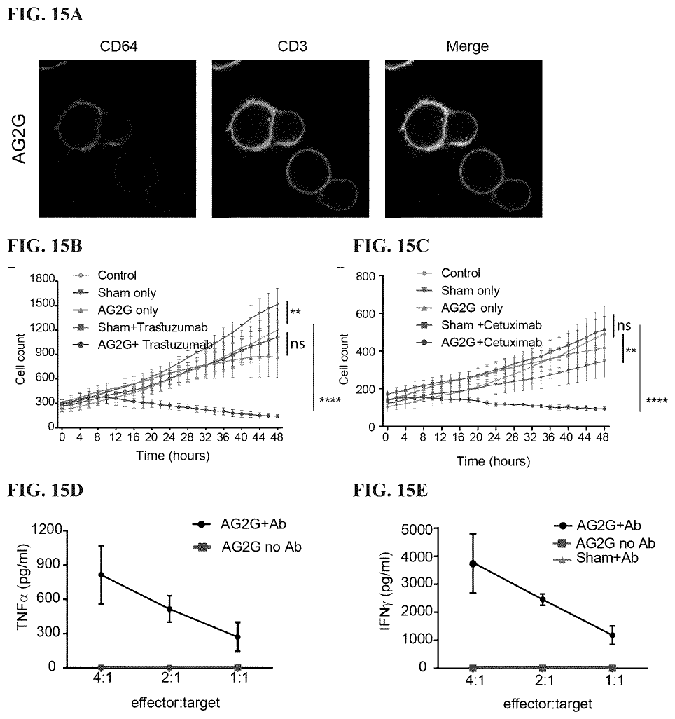

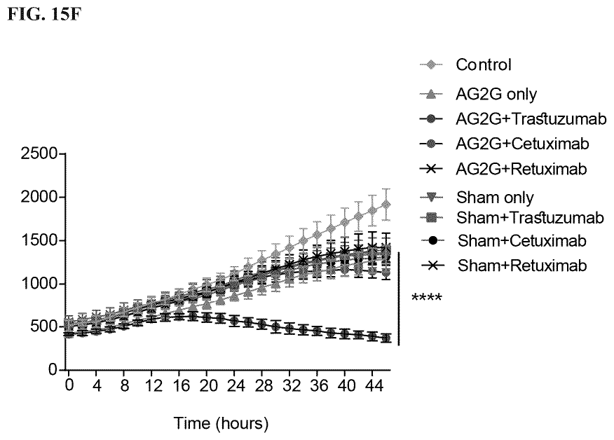

In the same manner, human T cells obtained from healthy donor PBMCs that were infected with Alpha-2A-Gamma or Alpha-gamma-2A-Gamma in pMIGII vector (FIGS. 14A-B) were co-cultured with HT-29 H2B-tdTomato target cells (4:1 effector:target ratio) that express HER2 or EGFR on their membrane (FIG. 14C) with an anti-HER2 antibody Trastuzumab (FIGS. 14E-F). Both Alpha-2A-Gamma and Alpha-gamma-2A-Gamma showed killing activity when Trastuzumab was added to the HER2-expressing cells (FIG. 14E) compared to the EGFR-expressing cells (FIG. 14F), in which no killing activity was observed. However, Alpha-gamma-2A-Gamma showed stronger activity than Alpha-2A-Gamma, as can be seen by the numbers of the live target cells along 48 hours co-culture. Next, human T cells were infected with retrovirus carrying Alpha-gamma-2A-Gamma in pMSVG.1 vector (FIG. 15A). The Alpha-gamma-2A-Gamma infected cells were compared for their killing activity to sham-infected cells, when added to HT-29 H2B tdTomato cells expressing HER2 with or without Trastuzumab (FIG. 15B) or HT-29 H2B tdTomato cells expressing EGFR with or without Cetuximab (FIG. 15C). In both cases Alpha-gamma-2A-Gamma and not sham-infected cells were able to elicit killing activity only when the tumor-binding antibody was added. In addition to the killing activity, the activity of the Alpha-gamma-2A-Gamma expressing T cells was evaluated by cytokines release. To this end, the Alpha-gamma-2A-Gamma or sham-infected cells were co-cultured with HT-29 H2B tdTomato expressing HER2 together with Trastuzumab in different effector:target ratios, and TNFα (FIG. 15D) and IFNγ (FIG. 15E) were measured by ELISA. The results show a correlation between the numbers of Alpha-gamma-2A-Gamma effector to target cells and the levels of cytokines released, while co-culturing sham-infected cells with the target cells did not result in cytokines release. Following, in order to evaluate the specificity of the Alpha-gamma-2A-Gamma activity to the antibody targeting the target cells, Alpha-gamma-2A-Gamma expressing human T cells were co-cultured with HT-29 H2B tdTomato HER2 expressing cells together with relevant or irrelevant antibodies (FIG. 15F). Following 48 hours of co-culture, only the HER-2 binding antibody, Trastuzumab, but not Cetuximab or Retuximab elicited killing of target cells. Taken together, these results further validate that concomitant signaling through FcγRI and the FcRγ signaling chain can exert killing capacities in T cells whenever the target cells are coated with antibodies; and that the Alpha-gamma-2A-Gamma construct is more potent and efficacious compared to the Alpha-2A-gamma construct.

### Example 2

### T Cells Exogenously Expressing FcRγ and FcγRI Fused to the Intracellular Domain of FcRγ Have In-Vivo Anti-Tumor Effects

To test the anti-tumor activity of T cells infected with Alpha-gamma-2A-Gamma in vivo, 2×10⁵ B16 cells were injected sub-cutaneously to C57BL/6 mice. Once tumors reached palpable size, mice were injected with 300 μg anti-TRP-1 antibody sub-cutaneously and 2×10⁶ CD3⁺ cells infected with Alpha-gamma-2A-Gamma intravenously. As controls, tumor bearing mice were either left untreated, treated with the antibody plus non-infected CD3+ T cells, or with Alpha-gamma-2A-Gamma infected CD3+ T cells without anti-TRP-1 antibody. The results showed no increase in tumor volume only in mice treated with both Alpha-gamma-2A-Gamma infected CD3+ T cells and anti-TRP-1 antibody (FIG. 12).

To demonstrate the anti-tumor activity of the T cells infected with Alpha-gamma-2A-Gamma in human subjects, PBMC are isolated from the apheresis product by density gradient with Ficoll-Hypaque (Lymphocyte Separation Medium, Axis-Shield Diagnostics, Scotland). Following, cells are cultured in a medium containing human serum, IL-2 and anti-CD3 monoclonal antibody OKT-3. Following few days of proliferation, the cells are transduced with the Alpha-gamma-2A-Gamma retroviral vector, followed by 1-2 weeks culturing of the transduced cells in complete medium supplemented with IL-2. These cell are then infused to a subject in need thereof in combination with an antibody targeting the pathologic cells.

### Example 3

### Exogenous Expression of FcRγ and Anti-EGFR scFv Fused to the Intracellular Domain of FcRγ in CD4+ and CD8+ T Cells Induces Effective Tumor Cell Lysis

The following construct is cloned: an anti-EGFR scFv fused to the extracellular D3 domain and transmembrane domain of FcγRI fused to the intracellular domain of FcRγ, T2A skipping peptide and FcRγ (SEQ ID NOs: 37-38), denoted herein as scFv-alpha-gamma-2A-gamma (FIG. 13). The plasmids are packed into retroviruses, and used to infect CD4⁺ and CD8⁺ T cells. Following, the killing activity of the transduced T cells is evaluated in-vitro on EGFR expressing tumor cells, for examples H29 colon cancer cells which express histone H2B-tdTomato. Co-culture and killing is evaluated following imaging by incuCyte imager.

### Example 4

### Exogenous Expression of FcRγ and FcγRI Fused to the Intracellular Domain of FcRγ in γδT Cells and NK Cells

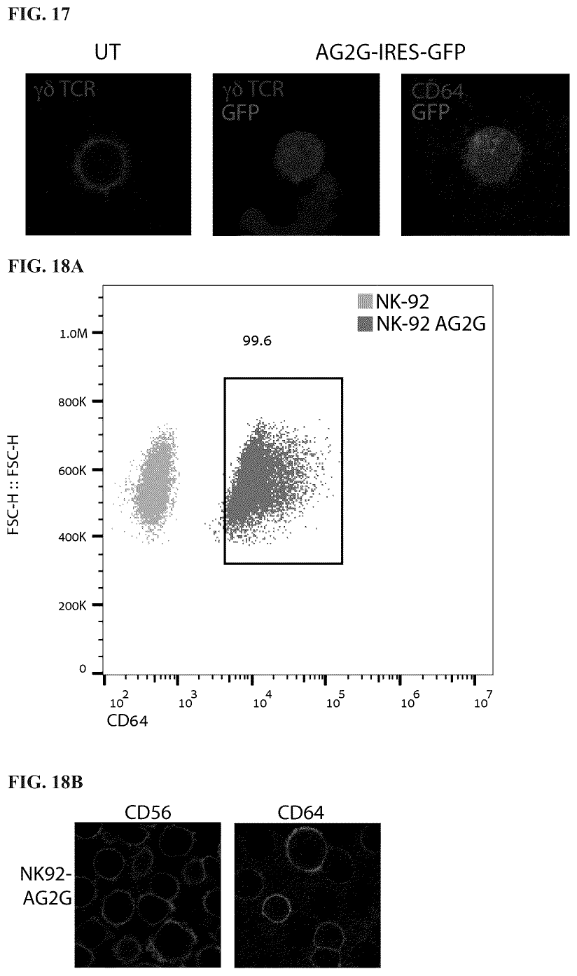

Effector cells other than αβ T cells, such as γδ T cells or NK cells can also be genetically-engineered to express Alpha-gamma-2A-Gamma. In order to demonstrate expression in γδ T cells, PBMC's from a healthy donor were incubated with IL-2 and Zoledronic acid in order to expand the γδ T cells population. Following 8 days in culture, cells were separated using EasySep™ Human Gamma/Delta T Cell Isolation Kit, and transduced with Alpha-gamma-2A-Gamma pMIGII construct. The infected cells were imaged by confocal microscopy (FIG. 17). γδ T cells were identified by the specific anti-γδ TCR antibody, and only the transduced cells showed GFP expression originated from the pMIGII vector. The GFP expression was corolated with CD64 staining on the cell membrane. In order to demonstrate expression in NK cells, NK-92 cells were infected with retrovirus carrying Alpha-gamma-2A-Gamma in pMSVG.2 vector. Only infected cells showed high levels of CD64 in flow cytometry analysis (FIG. 18A). Following, the cells were stained for the NK marker CD56 and for CD64 originated from the Alpha-gamma-2A-Gamma and were imaged by confocal microscopy (FIG. 18B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1.-20. (canceled)

21. A method of treating a disease associated with a pathologic cell in a subject in need thereof, the method comprising:
administering to the subject a composition comprising a therapeutically effective amount of an engineered immune cell comprising a multi subunit protein comprising at least three exogenously-expressed cell membrane polypeptides each comprising an amino acid sequence of an Fc receptor common γ chain (FcRγ), wherein the amino acid sequence is capable of transmitting an activating signal and wherein at least one but not all of the at least three polypeptides comprises an extracellular binding domain capable of binding a target on the pathologic cell such that upon binding the activating signal is transmitted in the engineered immune cell, wherein the pathologic cell presents the target on the cell surface, thereby enabling transmission of the activating signal in the engineered immune cell and treating the disease in the subject.

22. The method of claim 21, wherein the extracellular binding domain is of an antibody and the target is an antigen.

23. The method of claim 21, wherein the composition further comprises a therapeutically effective amount of the target, or wherein the subject is treated with an additional therapeutic composition comprising the target, the additional therapeutic composition being specific for the pathologic cell.

24. The method of claim 21, wherein the engineered immune cell comprises a T cell or an NK cell.

25. The method of claim 22, wherein the composition comprises an antibody.

26. The method of claim 25, wherein the antibody comprises an IgG.

27. The method of claim 25, wherein the antibody is an anti-CD44 antibody, an anti-TRP-1 antibody, an anti-PDL-1 antibody, an anti-CD20 antibody, an anti-HER-2 antibody.

28. The method of claim 25, wherein the antibody binds EGFR, TRP-1, CD44, PDL-1, HER-2, MUC-1, MUC-16, CEA or EpCAM.

29. The method of claim 21, wherein the pathologic cell is a cancerous cell, and wherein the disease comprises cancer.

30. The method of claim 29, wherein the cancer or the cancerous cell expresses a marker selected from the group consisting of PDL-1, E-Cadherin, CD19, MUC1, TRP-1 and TRP-2.

31. The method of claim 29, wherein the cancer comprises lymphoma, melanoma, colon cancer, breast cancer, or pancreatic cancer.

32. The method of claim 21, wherein the disease is an infection.

33. The method of claim 32, wherein the infection comprises a viral infection.

34. The method of claim 33, wherein the viral infection comprises human immunodeficiency virus (HIV)-induced acquired immunodeficiency syndrome (AIDS), influenza, rhinoviral infection, viral meningitis, Epstein-Barr virus (EBV) infection, hepatitis A, B or C virus infection, measles, papilloma virus infection/warts, cytomegalovirus (CMV) infection, Herpes simplex virus infection, yellow fever, Ebola virus infection, or rabies.

35. The method of claim 32, wherein the infection comprises a bacterial infection.

36. The method of claim 21, wherein the extracellular binding domain is of an Fcγ receptor and the target is an Fc ligand.

37. The method of claim 36, wherein the Fcγ receptor is CD64.

38. The method of claim 21, wherein one or more of the cell membrane polypeptides comprises a dimerizing moiety.

39. The method of claim 38, wherein the dimerizing moiety comprises an amino acid sequence of a transmembrane domain of the FcRγ.

40. The method of claim 21, wherein the activating signal comprises an ITAM motif.

41. The method of claim 21, wherein the FcRγ amino acid sequence is full length FcRγ.

42. The method of claim 21, wherein the FcRγ amino acid sequence comprises a FcRγ fragment.


SEQUENCE LISTING (77 sequences)

SEQ ID NO: 1 - protein, 508 aa, synthetic construct
  REGION 1..508: note = Alpha-2A-gamma construct aa sequence (mouse origin)
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQSEGRGSLLTCGDVEENPGPMISAVILFLLLLVEQAAALGEPQLCYILDAVLFLYGIVLTLLYCRLKIQVRKAAIASREKADAVYTGLNTRSQETYETLKHEKPPQ

SEQ ID NO: 2 - DNA, 1527 bp, synthetic construct
  misc_feature 1..1527: note = Alpha-2A-gamma construct NA sequence (mouse origin)
  source 1..1527: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgaaaatacacaggctgcagagagagaagaaatacaacttagaagtccctttggtttctgagcagggaaagaaagcaaattcctttcagcaagttagaagcgatggcgtgtatgaagaagtaacagccactgcgagccagaccacaccaaaagaagcgcccgatggacctcgaagctcagtgggtgactgtggacccgagcagcctgaaccccttcctcccagtgacagtactggggcacaaacttcccaaagtgagggcagaggaagtctgctaacatgcggtgacgtcgaggagaatcctggcccaatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 3 - protein, 466 aa, synthetic construct
  REGION 1..466: note = Alpha-gamma 2A-gamma construct aa sequence (mouse origin)
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVRLKIQVRKAAIASREKADAVYTGLNTRSQETYETLKHEKPPQEGRGSLLTCGDVEENPGPMISAVILFLLLLVEQAAALGEPQLCYILDAVLFLYGIVLTLLYCRLKIQVRKAAIASREKADAVYTGLNTRSQETYETLKHEKPPQ

SEQ ID NO: 4 - DNA, 1398 bp, synthetic construct
  misc_feature 1..1398: note = Alpha-gamma 2A-gamma construct na sequence (mouse origin)
  source 1..1398: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgcgactcaagatccaggtccgaaaggcagctatagccagccgtgagaaagcagatgctgtctacacgggcctgaacacccggagccaggagacatatgagactctgaagcatgagaaaccaccccaggagggcagaggaagtctgctaacatgcggtgacgtcgaggagaatcctggcccaatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccag

SEQ ID NO: 5 - protein, 405 aa, synthetic construct
  REGION 1..405: note = Alpha-CD8-gamma construct aa sequence (mouse origin)
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPTTTKPVLRTPSPVHPTGTSQPQRPEDCRPRGSVKGTGLDFACDIYIWAPLAGICVALLLSLIITLIRLKIQVRKAAIASREKADAVYTGLNTRSQETYETLKHEKPPQ

SEQ ID NO: 6 - DNA, 1218 bp, synthetic construct
  misc_feature 1..1218: note = Alpha-CD8-gamma construct aa sequence (mouse origin)
  source 1..1218: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatccgactcaagatccaggtccgaaaggcagctatagccagccgtgagaaagcagatgctgtctacacgggcctgaacacccggagccaggagacatatgagactctgaagcatgagaaaccaccccagtag

SEQ ID NO: 7 - protein, 466 aa, synthetic construct
  REGION 1..466: note = Alpha-gamma-2A-Gamma(mut) construct AA sequence (mouse origin)
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVRLKIQVRKAAIASREKADAVYTGLNTRSQETYETLKHEKPPQEGRGSLLTCGDVEENPGPMISAVILFLLLLVEQAAALGEPQLGYILDAVLFLYGIVLTLLYGRLKIQVRKAAIASREKADAVYTGLNTRSQETYETLKHEKPPQ

SEQ ID NO: 8 - DNA, 1656 bp, synthetic construct
  misc_feature 1..1656: note = Alpha-gamma-2A-Gamma(mut) construct NA sequence (mouse origin)
  source 1..1656: mol_type = other DNA
atgattcttaccagctttggagatgacatgtggcttctaacaactctgctactttgggttccagtcggtggggaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgcgactcaagatccaggtccgaaaggcagctatagccagccgtgagaaagcagatgctgtctacacgggcctgaacacccggagccaggagacatatgagactctgaagcatgagaaaccaccccaggagggcagaggaagtctgctaacatgcggtgacgtcgaggagaatcctggcccaatgatctcagccgtgatcttgttcttgctccttttggtggaacaagcagccgccctgggagagccgcagctcggctatatcctggatgctgtcctgtttttgtatggtattgtccttaccctactctacggtcgactcaagatccaggtccgaaaggcagctatagccagccgtgagaaagcagatgctgtctacacgggcctgaacacccggagccaggagacatatgagactctgaagcatgagaaaccaccccagatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccag

SEQ ID NO: 9 - DNA, 37 bp, synthetic construct
  misc_feature 1..37: note = Single strand DNA oligonucleotide
  source 1..37: mol_type = other DNA
aataacactagtgccaccatgcctgaaccggcaaaat

SEQ ID NO: 10 - DNA, 33 bp, synthetic construct
  misc_feature 1..33: note = Single strand DNA oligonucleotide
  source 1..33: mol_type = other DNA
aacaaccccgggacttgtcgtcatcgtctttgt

SEQ ID NO: 11 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 12 - protein, 42 aa, Mus musculus
RLKIQVRKAAIASREKADAVYTGLNTRSQETYETLKHEKPPQ

SEQ ID NO: 13 - DNA, 126 bp, Mus musculus
  source 1..126: mol_type = genomic DNA
cgactcaagatccaggtccgaaaggcagctatagccagccgtgagaaagcagatgctgtctacacgggcctgaacacccggagccaggagacatatgagactctgaagcatgagaaaccaccccag

SEQ ID NO: 14 - protein, 273 aa, Mus musculus
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAP

SEQ ID NO: 15 - DNA, 819 bp, Mus musculus
  source 1..819: mol_type = genomic DNA
gaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcct

SEQ ID NO: 16 - protein, 23 aa, Mus musculus
VWFHILFYLSVGIMFSLNTVLYV

SEQ ID NO: 17 - DNA, 69 bp, Mus musculus
  source 1..69: mol_type = genomic DNA
gtctggtttcacatcctgttttatctgtcagtgggaataatgttttcgttgaacacggttctctatgtg

SEQ ID NO: 18 - protein, 296 aa, Mus musculus
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYV

SEQ ID NO: 19 - protein, 338 aa, Mus musculus
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVRLKIQVRKAAIASREKADAVYTGLNTRSQETYETLKHEKPPQ

SEQ ID NO: 20 - DNA, 1014 bp, Mus musculus
  source 1..1014: mol_type = genomic DNA
gaagtggttaatgccaccaaggctgtgatcaccttgcagcctccatgggtcagtattttccagaaggaaaatgtcactttatggtgtgaggggcctcacctgcctggagacagttccacacaatggtttatcaacggaacagccgttcagatctccacgcctagttatagcatcccagaggccagttttcaggacagtggcgaatacaggtgtcagataggttcctcaatgccaagtgaccctgtgcagttgcaaatccacaatgattggctgctactccaggcctcccgcagagtcctcacagaaggagaacccctggccttgaggtgtcacggatggaagaataaactggtgtacaatgtggttttctatagaaatggaaaatcctttcagttttcttcagattcggaggtcgccattctgaaaaccaacctgagtcacagcggcatctaccactgctcaggcacgggaagacaccgctacacatctgcaggagtgtccatcacggtgaaagagctgtttaccacgccagtgctgagagcatccgtgtcatctcccttcccggaggggagtctggtcaccctgaactgtgagacgaatttgctcctgcagagacccggcttacagcttcacttctccttctacgtgggcagcaagatcctggagtacaggaacacatcctcagagtaccatatagcaagggcggaaagagaagatgctggattctactggtgtgaggtagccacggaggacagcagtgtccttaagcgcagccctgagttggagctccaagtgcttggtccccagtcatcagctcctgtctggtttcacatcctgttttatctgtcagtgggaataatgttttcgttgaacacggttctctatgtgcgactcaagatccaggtccgaaaggcagctatagccagccgtgagaaagcagatgctgtctacacgggcctgaacacccggagccaggagacatatgagactctgaagcatgagaaaccaccccag

SEQ ID NO: 21 - protein, 21 aa, Mus musculus
LCYILDAVLFLYGIVLTLLYC

SEQ ID NO: 22 - DNA, 63 bp, Mus musculus
  source 1..63: mol_type = genomic DNA
ctctgctatatcctggatgctgtcctgtttttgtatggtattgtccttaccctactctactgt

SEQ ID NO: 23 - protein, 63 aa, Mus musculus
LCYILDAVLFLYGIVLTLLYCRLKIQVRKAAIASREKADAVYTGLNTRSQETYETLKHEKPPQ

SEQ ID NO: 24 - DNA, 189 bp, Mus musculus
  source 1..189: mol_type = genomic DNA
ctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccag

SEQ ID NO: 25 - protein, 36 aa, synthetic construct
  REGION 1..36: note = OX40 intracellular domain AA sequence
RKAWRLPNTPKPCWGNSFRTPIQEEHTDAHFTLAKI

SEQ ID NO: 26 - protein, 48 aa, synthetic construct
  REGION 1..48: note = 41BB intracellular domain AA sequence
SVLKWIRKKFPHIFKQPFKKTTGAAQEEDACSCRCPQEEEGGGGGYEL

SEQ ID NO: 27 - protein, 85 aa, synthetic construct
  REGION 1..85: note = IL-2R common gamma intracellular AA sequence
ERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 28 - protein, 186 aa, synthetic construct
  REGION 1..186: note = TNFR2 intracellular signaling domain AA SEQUENCE
KKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 29 - protein, 147 aa, synthetic construct
  REGION 1..147: note = IL-12Rb1 intracellular AA SEQUENCE
NRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 30 - protein, 216 aa, synthetic construct
  REGION 1..216: note = IL-12Rb2 intracellular AA SEQUENCE
RYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 31 - protein, 249 aa, synthetic construct
  REGION 1..249: note = IL-23R intracellular AA SEQUENCE
NRSLRIGIKRKVLLMIPKWLYEDIPNMENSNVAKLLQEKSVFENDNASEQALYVDPVLTEISEISPLEHKPTDYKEERLTGLLETRDCPLGMLSTSSSVVYIPDLNTGYKPQVSNVPPGGNLFINRDERDPTSLETTDDHFARLKTYPNFQFSASSMALLNKTLILDELCLVLNQGEFNSLDIKNSRQEETSIVLQSDSPSETIPAQTLLSDEFVSCLAIGNEDLPSINSYFPQNVLESHFSRISLFQK

SEQ ID NO: 32 - protein, 202 aa, synthetic construct
  REGION 1..202: note = IFNgR1 intracellular AA SEQUENCE
WYTKNNSFKRKSIMLPKSLLSVVKSATLETKPESKYSLVTPHQPAVLESETVICEEPLSTVTAPDSPEAAEQEELSKETKALEAGGSTSAMTPDSPPTPTQRRSFSLLSSNQSGPCSLTAYHSRNGSDSGLVGSGSSISDLESLPNNNSETKMAEHDPPPVRKAPMASGYDKPHMLVDVLVDVGGKESLMGYRLTGEAQELS

SEQ ID NO: 33 - protein, 67 aa, synthetic construct
  REGION 1..67: note = IFNgR2 intracellular AA SEQUENCE
LKYQSRVKYWFQAPPNIPEQIEEYLKDPDQFILEVLDKDGSPKEDSWDSVSIISSPEKERDDVLQTP

SEQ ID NO: 34 - protein, 271 aa, synthetic construct
  REGION 1..271: note = IL-2Rb intracellular AA SEQUENCE
KCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 35 - protein, 217 aa, synthetic construct
  REGION 1..217: note = IL-1R1 intracellular AA sequence
KVFKVDIVLWYRDSCSGFLPSKASDGKTYDAYILYPKTLGEGSFSDLDTFVFKLLPEVLEGQFGYKLFIYGRDDYVGEDTIEVTNENVKKSRRLIIILVRDMGGFSWLGQSSEEQIAIYNALIQEGIKIVLLELEKIQDYEKMPDSIQFIKQKHGVICWSGDFQERPQSAKTRFWKNLRYQMPAQRRSPLSKHRLLTLDPVRDTKEKLPAATHLPLG

SEQ ID NO: 36 - protein, 182 aa, synthetic construct
  REGION 1..182: note = IL-1AcP intracellular AA sequence
YRAHFGTDETILDGKEYDIYVSYARNVEEEEFVLLTLRGVLENEFGYKLCIFDRDSLPGGIVTDETLSFIQKSRRLLVVLSPNYVLQGTQALLELKAGLENMASRGNINVILVQYKAVKDMKVKELKRAKTVLTVIKWKGEKSKYPQGRFWKQLQVAMPVKKSPRWSSNDKQGLSYSSLKNV

SEQ ID NO: 37 - protein, 526 aa, synthetic construct
  REGION 1..526: note = scFv-alpha-gamma-2A-gamma construct aa sequence (human origin)
MALPVTALLLPLALLLHAARPQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASGGGGSGGGGSGGGGSILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRLKIQVRKAAITSYEKSDGVYTGLSTRNQETYETLKHEKPPQEGRGSLLTCGDVEENPGPMIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 38 - DNA, 1578 bp, synthetic construct
  misc_feature 1..1578: note = scFv-alpha-gamma-2A-gamma construct NA sequqence (human origin)
  source 1..1578: mol_type = other DNA
atggccttaccagtgaccgccttgctcctgccgctggccttgctgctccacgccgccaggccgcaagtacaacttaaacagagcggtcctggtctggtgcaaccctcccaaagcctttcaatcacgtgtaccgtttccgggttttcacttacgaattacggcgtccattgggtgcggcaaagtccaggcaaaggtctggagtggcttggcgtaatctggtcaggaggtaatacggactacaacacaccttttactagcagacttagcatcaacaaggataacagtaagagtcaggtcttcttcaagatgaatagtttgcaatccaatgatacagctatctactactgcgccagagctttgacctactacgattatgagtttgcatattggggtcaaggaactctggtaacagtaagcgccgcttcaggtggaggtggttctgggggtgggggctcaggtgggggaggtagcattcttctcacccagtcacccgttatactttccgtatctccgggagaaagggtctctttcagttgtcgcgcgagtcaaagcataggcactaatatccactggtaccaacaaagaacaaacggttcacccagactgttgatcaaatatgcgtcagagagtattagcgggatccctagcagattttccgggagcggaagcggtactgattttacactgagcattaatagcgttgaaagtgaggatattgcggactactactgccaacaaaacaataactggcctacaactttcggtgcaggtacgaaattggagctcgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgactgaagatccaagtgcgaaaggcagctataaccagctatgagaaatcagatggtgtttacacgggcctgagcaccaggaaccaggagacttacgagactctgaagcatgagaaaccaccacaggagggcagaggaagtctgctaacatgcggtgacgtcgaggagaatcctggcccaatgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 39 - protein, 42 aa, Homo sapiens
RLKIQVRKAAITSYEKSDGVYTGLSTRNQETYETLKHEKPPQ

SEQ ID NO: 40 - DNA, 126 bp, Homo sapiens
  source 1..126: mol_type = genomic DNA
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 41 - protein, 121 aa, synthetic construct
  REGION 1..121: note = anti-EFGR variable heavy chain aa sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAAS

SEQ ID NO: 42 - protein, 105 aa, synthetic construct
  REGION 1..105: note = anti-EFGR variable light chain aa sequence
ILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLEL

SEQ ID NO: 43 - protein, 241 aa, synthetic construct
  REGION 1..241: note = Anti EGFR scFv aa sequence
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASGGGGSGGGGSGGGGSILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLEL

SEQ ID NO: 44 - protein, 277 aa, Homo sapiens [sequence omitted]

SEQ ID NO: 45 - protein, 180 aa, Mus musculus
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLR

SEQ ID NO: 46 - protein, 180 aa, Homo sapiens
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLN

SEQ ID NO: 47 - protein, 21 aa, Homo sapiens
VLFYLAVGIMFLVNTVLWVTI

SEQ ID NO: 48 - protein, 298 aa, Homo sapiens
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTI

SEQ ID NO: 49 - protein, 340 aa, Homo sapiens
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRLKIQVRKAAITSYEKSDGVYTGLSTRNQETYETLKHEKPPQ

SEQ ID NO: 50 - protein, 21 aa, Homo sapiens
LCYILDAILFLYGIVLTLLYC

SEQ ID NO: 51 - protein, 63 aa, Homo sapiens
LCYILDAILFLYGIVLTLLYCRLKIQVRKAAITSYEKSDGVYTGLSTRNQETYETLKHEKPPQ

SEQ ID NO: 52 - protein, 401 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASGGGGSGGGGSGGGGSILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRLKIQVRKAAITSYEKSDGVYTGLSTRNQETYETLKHEKPPQ

SEQ ID NO: 53 - protein, 20 aa, synthetic construct
  REGION 1..20: note = Linker amino acid sequence
  REPEAT 1..5: note = repeat sequence 1-4 times
  REPEAT 6..10: note = may be absent
  REPEAT 11..15: note = may be absent
  REPEAT 16..20: note = may be absent
GGGGSGGGGSGGGGSGGGGS

SEQ ID NO: 54 - protein, 9 aa, synthetic construct
  REGION 1..9: note = Linker amino acid sequence
GGGGSGGGG

SEQ ID NO: 55 - protein, 8 aa, synthetic construct
  REGION 1..8: note = Linker amino acid sequence
GGGGGGGG

SEQ ID NO: 56 - protein, 6 aa, synthetic construct
  REGION 1..6: note = Linker amino acid sequence
GGGGGG

SEQ ID NO: 57 - protein, 15 aa, synthetic construct
  REGION 1..15: note = Linker amino acid sequence
  REPEAT 1..5: note = Repeat 1-3 times
  REPEAT 6..10: note = may be absent
  REPEAT 11..15: note = may be absent
EAAAKEAAAKEAAAK

SEQ ID NO: 58 - protein, 5 aa, synthetic construct
  REGION 1..5: note = Linker amino acid sequence
PAPAP

SEQ ID NO: 59 - protein, 15 aa, synthetic construct
  REGION 1..15: note = Linker amino acid sequence
GGGGSGGGGSGGGGS

SEQ ID NO: 60 - protein, 93 aa, Mus musculus
ASVSSPFPEGSLVTLNCETNLLLQRPGLQLHFSFYVGSKILEYRNTSSEYHIARAEREDAGFYWCEVATEDSSVLKRSPELELQVLGPQSSAP

SEQ ID NO: 61 - protein, 97 aa, Homo sapiens
ASVTSPLLEGNLVTLSCETKLLLQRPGLQLYFSFYMGSKTLRGRNTSSEYQILTARREDSGLYWCEAATEDGNVLKRSPELELQVLGLQLPTPVWFH

SEQ ID NO: 62 - DNA, 1395 bp, synthetic construct
  misc_feature 1..1395: note = Alpha-2A-gamma construct NA sequqence (HUMAN origin)
  source 1..1395: mol_type = other DNA
atggccttaccagtgaccgccttgctcctgccgctggccttgctgctccacgccgccaggccgcaagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaactctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgactgaagatccaagtgcgaaaggcagctataaccagctatgagaaatcagatggtgtttacacgggcctgagcaccaggaaccaggagacttacgagactctgaagcatgagaaaccaccacaggagggcagaggaagtctgctaacatgcggtgacgtcgaggagaatcctggcccaatgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 63 - protein, 465 aa, synthetic construct
  REGION 1..465: note = Alpha-gamma 2A-gamma construct aa sequence (HUMAN origin)
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRLKIQVRKAAITSYEKSDGVYTGLSTRNQETYETLKHEKPPQEGRGSLLTCGDVEENPGPMIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 64 - protein, 277 aa, synthetic construct [sequence omitted]

SEQ ID NO: 65 - DNA, 831 bp, synthetic construct
  misc_feature 1..831: note = Extracellular domain of CD64 (D1+D2+D3) NA sequence (HUMAN)
  source 1..831: mol_type = other DNA
caagtggacaccacaaaggcagtgatcactttgcagcctccatgggtcagcgtgttccaagaggaaaccgtaaccttgcattgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaactctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcat

SEQ ID NO: 66 - DNA, 63 bp, synthetic construct
  misc_feature 1..63: note = Transmembrane domain of CD64 NA sequence (HUMAN)
  source 1..63: mol_type = other DNA
gtccttttctatctggcagtgggaataatgtttttagtgaacactgttctctgggtgacaata

SEQ ID NO: 67 - DNA, 63 bp, synthetic construct
  misc_feature 1..63: note = FcR gamma chain TM nA SEQUENCE (HUMAN)
  source 1..63: mol_type = other DNA
ctctgctatatcctggatgccatcctgtttctgtatggaattgtcctcaccctcctctactgt

SEQ ID NO: 68 - DNA, 15 bp, synthetic construct
  misc_feature 1..15: note = FcR gamma chain EC NA SEQUENCE (HUMAN)
  source 1..15: mol_type = other DNA
ctgggagagcctcag

SEQ ID NO: 69 - protein, 5 aa, synthetic construct
  REGION 1..5: note = FcR gamma chain EC AA SEQUENCE (HUMAN)
LGEPQ

SEQ ID NO: 70 - DNA, 204 bp, synthetic construct
  misc_feature 1..204: note = FcR gamma chain EC+TM+IC NA SEQUENCE (HUMAN)
  source 1..204: mol_type = other DNA
ctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 71 - protein, 68 aa, synthetic construct
  REGION 1..68: note = FcR gamma chain EC+TM+IC AA SEQUENCE (HUMAN)
LGEPQLCYILDAILFLYGIVLTLLYCRLKIQVRKAAITSYEKSDGVYTGLSTRNQETYETLKHEKPPQ

SEQ ID NO: 72 - DNA, 63 bp, synthetic construct
  misc_feature 1..63: note = CD8A signal peptide NA
  source 1..63: mol_type = other DNA
atggccttaccagtgaccgccttgctcctgccgctggccttgctgctccacgccgccaggccg

SEQ ID NO: 73 - protein, 21 aa, synthetic construct
  REGION 1..21: note = CD8A signal peptide AA
MALPVTALLLPLALLLHAARP

SEQ ID NO: 74 - DNA, 54 bp, synthetic construct
  misc_feature 1..54: note = SIGNAL SEQUENCE NA
  source 1..54: mol_type = other DNA
atgattccagcagtggtcttgctcttactccttttggttgaacaagcagcggcc

SEQ ID NO: 75 - protein, 18 aa, synthetic construct
  REGION 1..18: note = SIGNAL SEQUENCE AA
MIPAVVLLLLLLVEQAAA

SEQ ID NO: 76 - DNA, 54 bp, synthetic construct
  misc_feature 1..54: note = T2A peptide NA
  source 1..54: mol_type = other DNA
gagggcagaggaagtctgctaacatgcggtgacgtcgaggagaatcctggccca

SEQ ID NO: 77 - protein, 18 aa, synthetic construct
  REGION 1..18: note = T2A peptide AA
EGRGSLLTCGDVEENPGP